(19) United States
(12) Patent Application Publication
(10) Pub. No.: US 2004/0260234 A1
(43) Pub. Date: Dec. 23, 2004

(54) Apparatus and methods for repetitive microjet durg delivery priority statement

(76) Inventors: Ravi Srinivasan, San Jose, CA (US); Ruben Rathnasingham, San Mateo, CA (US); Errol Bernard Arkilic, Arlington, VA (US)

Correspondence Address: MORGAN, LEWIS & BOCKIUS, LLP., 2 PALO ALTO SQUARE, 3000 EL CAMINO REAL, PALO ALTO, CA, 94306, US

(21) Appl. No.: 10/829,888

(22) Filed: Apr. 21, 2004

Related U.S. Application Data
(60) Provisional application No. 60/463,905, filed on Apr. 21, 2003.
(60) Provisional application No. 60/483,604, filed on Jun. 30, 2003.
(60) Provisional application No. 60/492,342, filed on Aug. 5, 2003.

(51) Int. Cl. A61M 31/00

(52) U.S. Cl. 604/66; 604/890.1; 604/70; 600/309

(57) ABSTRACT

An active, transdermal delivery system includes a support structure and a fluid reservoir within the support structure configured to contain a fluid to be delivered transdermally. There is also at least one exit orifice defined in the support structure that is in communication with the fluid reservoir. The orifice has a diameter of between about 1 μm and 500 μm. Furthermore, a repeatable activation means is disposed within the support structure and is in cooperation with the exit orifice for ejection of fluid in response to an activation signal.

### PRIORITY STATEMENT

This application claims priority to provisional application Nos. 60/463,905, filed Apr. 21, 2003; 60/483,604, filed Jun. 30, 2003; and 60/492,342 filed Aug. 05, 2003; each of which is incorporated herein by reference in their entirety.

### FIELD OF THE INVENTION

Generally the present invention relates to the field of drug delivery. More particularly, the present invention provides a device and methods for sustained transdermal drug delivery using repetitive microjets.

### BACKGROUND OF THE INVENTION

Traditionally, the dominant method of delivering medication into the human body has been by oral ingestion of pills. Once ingested, the medication is theoretically absorbed across the gastrointestinal (GI) tract and into the blood stream for systemic delivery. However, a large fraction of drug candidates, which may be highly promising drugs, either do not have the right solubility to be absorbed by the GI tract or are destroyed by digestive secretions prior to being absorbed. Of the drugs that are absorbed by the GI tract, a large fraction of these are metabolized by the liver and rendered inactive before their full beneficial effect can be appreciated. Furthermore, today's pharmaceutical industry is shifting toward higher molecular weight biopharmaceutical type drugs. Along with this shift will come an increase in the number of drugs that cannot effectively be delivered orally.

Another method of drug delivery is transdermal drug delivery. Transdermal drug delivery is the delivery of the drug substance directly across the skin barrier. Transdermal drug delivery has been in existence for roughly two decades. Transdermal delivery has many advantages over other drug delivery methods, including avoiding first pass metabolism and the ability to maintain consistent systemic dosage levels avoiding the peaks and troughs experienced with pills, injections, pulmonary, and transmucosal drug delivery methods. Furthermore, transdermal drug delivery is an extremely convenient dosage vehicle for the patient and tends to achieves high levels of patient compliance.

While applications proving appropriate for transdermal delivery are highly effective, few drug candidates actually materialize as candidates for transdermal delivery. Traditional transdermal drug delivery relies on the drug permeating the skin. In use, only a small number of drugs are actually passively absorbed through the skin at therapeutic levels. Currently, there are approximately only ten drugs that are commercially available in transdermal formats. Moreover, today's macromolecule drugs, have a much larger mass than the typical successful transdermal drug and have limited solubility in lipid bilayers and, therefore, will have even more limited transdermal applications.

The main barrier to diffusion of pharmaceuticals across the skin is the outermost layer of the skin, the stratum corneum. The stratum corneum consists of densely packed keratinocytes (flat dead cells filled with keratin fibers) surrounded by highly ordered lipid bilayers, creating an effective barrier to permeability. Directly beneath the stratum corneum is the epidermis. The epidermis is rich in cells of the immune system, and therefore a target for drug delivery for therapies that are directed to or involve the immune system. Beneath the epidermis is the dermis. The dermis has a rich network of blood capillaries and, therefore, is an attractive target for systemic drug delivery since drugs presented to the capillary network rapidly enter the circulatory system and are systemically delivered throughout the body.

Various methods for enhancing transdermal drug delivery across the stratum corneum have been devised including utilizing enhancing agents or stimulants such as chemical, voltage charge, ultrasonic waves, thermal treatments, microneedles, and laser assist techniques. For example, see U.S. Pat. No's. 6,352,506 and 6,216,033. However, the development and broad acceptance of these methods has been hampered by skin irritation, incompatibility with the drug formulations, and the complexity and expense of the devices themselves. Furthermore, these techniques do not offer the capability of time-dependent dosage delivery, which is crucial to many therapeutics, including insulin.

Another mechanism of drug delivery is the use of needless injections or high-speed jet injectors. High-speed jet injectors have been utilized as hypodermic syringe replacements for many years. For example, see U.S. Pat. No's. 2,380,534; 4,596,556; 5,520,639; 5,630,796 and 5,993,412. Jet injectors move the solution to be injected at a high rate of speed and eject the solution as a jet, penetrating the stratum corneum and depositing the solution into the dermis and subcutaneous regions of the skin.

While traditional high-speed jets are capable of transporting drugs across the stratum corneum, a drawback of this mechanism is that they deliver a large quantity of the composition being delivered in a one-time jet injection. As a result, some of the drug is often forced back out of the penetration pore from the pressure that is developed by the large quantity of the delivery. Moreover, the one-time delivery fails to maintain a sustained systemic drug concentration at therapeutic levels. Still further, due to the large quantity of drug delivered at one-time, patients often experience skin irritation, pain, swelling, and other undesirable effects similar to injections with hypodermic syringes.

Therefore, less-invasive techniques for sustained transdermal delivery of a composition at consistent therapeutic levels to a patient would be highly desirable.

### SUMMARY OF THE INVENTIO

The present invention provides an active, fluid delivery system that generally includes a support structure with at least one exit orifice. The exit orifice has a diameter of between about 1 μm and about 500 μm. The fluid delivery system also has a fluid reservoir configured to contain a fluid to be delivered to a tissue. The fluid reservoir is configured and dimensioned to communicate with the exit orifice. A repeatable activation means cooperates with the fluid reservoir and the exit orifice for ejecting fluid in response to an activation signal.

In an alternative embodiment, the fluid reservoir and the repeatable activation means are disposed in the support structure. The support structure can be adapted to be in contact with a skin surface with the exit orifice adjacent the skin surface. The support structure can also include a nozzle defining the orifice. The nozzle is configured and dimensioned to accelerate the fluid exiting therefrom.

According to another embodiment of the present invention, the fluid delivery system includes a controller in communication with the repeatable activation means. The controller is designed to be capable of generating the activation signal. The controller can be a microprocessor that is programmable to control a patterned administration regime to be delivered from the fluid delivery system. The patterned administration regime preferably occurs over a time period of not less than about 500 ms and not more than about 10 days.

The nozzle of the fluid delivery system can be configured to maintain the fluid remote from the tissue a substantially fixed distance prior to ejection of the fluid from the nozzle. The fixed distance preferably spaces the fluid, prior to ejection of the fluid, not more than about 5000 μm from the tissue.

According to yet another embodiment, the fluid delivery system includes an array of exit orifices defined in the support structure and in communication with the fluid reservoir. The fluid reservoir can include a storage reservoir configured to store fluid. The fluid reservoir can also include a pressurization mechanism for pressurizing the stored fluid in the storage reservoir. Furthermore, the storage reservoir can be divided into at least two storage reservoirs by a reservoir divider.

According to an embodiment, there are at least two exit orifices defined in the support structure. A first exit orifice is in communication with a first storage reservoir storing a first fluid such that the first fluid can be ejected through the first exit orifice. There is also a at least a second exit orifice in communication with at least a second storage reservoir storing a second fluid such that the second fluid can be ejected through the second exit orifice.

According to an alternative embodiment, the reservoir divider can include a reservoir divider disruption mechanism configured and dimensioned to disrupt the reservoir divider prior to administration of a substance contained in the reservoir. The reservoir divider disruption mechanism can be a piezoelectric mechanism, for example.

In another embodiment, the fluid delivery system includes a sensor for sensing if a condition is satisfied. Also included is a control unit configured to produce the activation signal to actuate the repeatable activation means upon receiving a signal from the sensor that the condition is or is not satisfied. The sensor can be located remotely from the support structure, implanted into the patient, located within the support structure, or the like. Furthermore, the sensor is capable of sensing a biological condition of a patient, such as temperature, pressure, chemical or molecular concentration, or the like.

In yet another alternative embodiment, the fluid delivery device includes an antagonist reservoir configured and dimensioned in cooperation with the fluid reservoir such that, upon compromise of the integrity of both reservoirs, the antagonist reservoir releases an antagonist agent which can inactivate the fluid.

In a preferred embodiment, the fluid delivery system also includes a power supply for supplying a drive force for the activation signal and a drive force for the repeatable activation means.

According to an embodiment, the repeatable activation means is a piezoelectric mechanism that generates a pressure change in the fluid. According to another embodiment, the repeatable activation means is a phase change mechanism that generates a pressure change in the fluid. In yet another embodiment, the repeatable activation means is an electromagnetic mechanism that generates a pressure change in the fluid. In still another embodiment, the repeatable activation means is a high pressure hydraulic mechanism that generates a pressure change in the fluid. According to yet another embodiment, the repeatable activation means includes multiple explosive mechanisms, each explosive mechanism capable of generating a pressure change in the fluid upon detonation of said explosive mechanism.

According to a preferred embodiment, the repeatable activation means generates a pulse width of not less than about 5 ns and not more than about 10 μs in duration. The frequency of the repeatable activation means and a duty cycle and length of ejection of fluid are controlled by a control unit.

In a preferred embodiment, the system further includes a user interface in communication with the repeatable activation means. The user interface being configured to initiate the activation signal in response to manipulation of the user interface.

In use of an embodiment of the fluid delivery system, the fluid is to be delivered transdermally across epithelial tissue.

The fluid delivery system preferably includes a memory for storing a delivery profile and delivery history of the fluid delivered to the tissue.

In an alternative embodiment of the present invention, the fluid includes an analyte for delivery to the tissue and subsequent diagnoses of a biological condition.

According to an embodiment of the present invention including a phase change mechanism, the system further includes a flexible membrane dividing the fluid reservoir into a first compartment and a second compartment, wherein the first compartment contains an actuation fluid in communication with said phase change mechanism and the second compartment contains the fluid to be delivered. In yet another embodiment, the actuation fluid is positioned near the phase change mechanism and the actuation fluid is immiscible with the fluid to be delivered.

According to an embodiment of the present invention, the fluid ejection chamber, at least one exit orifice, and activation means are configured and dimensioned together for continuously cyclic repeatable ejection of fluid in the range of about 1 pl to about 800 nl.

### BRIEF DESCRIPTION OF THE FIGURES

For a better understanding of the nature and objects of the invention, reference should be made to the following detailed description, read in conjunction with the accompanying drawings, in which:

FIG. 1 is a schematic view of an embodiment of a repetitive microjet device according to an embodiment of the present invention;

FIG. 2A is a schematic view of an embodiment of a repetitive microjet device having an array of microjets according to the present invention;

FIG. 2B is a schematic view of another embodiment of a repetitive microjet device having an array of microjets according to the present invention;

FIG. 3 is a schematic view of another embodiment of a repetitive microjet device according to the present invention;

FIG. 4 is a schematic view of yet another embodiment of a repetitive microjet device according to the present invention;

FIG. 5 is a schematic view of still another embodiment of a repetitive microjet device according to the present invention;

FIG. 6 is a schematic view of a further embodiment of a repetitive microjet device according to the present invention;

FIG. 7 is a schematic view of a yet a further embodiment of a repetitive microjet device according to the present invention;

FIG. 8 is a schematic view of another embodiment of a repetitive microjet device according to the present invention;

FIG. 9 is a schematic view of another embodiment of a repetitive microjet device having an array of microjets according to the present invention;

FIG. 10 is a schematic view of an embodiment of a repetitive microjet device having a piezoelectric mechanism according to the present invention;

FIG. 11 is a schematic view of yet another embodiment of a repetitive microjet device having a piezoelectric mechanism according to the present invention;

FIG. 12 is a schematic view of another embodiment of a repetitive microjet device having an array of piezoelectric mechanism microjets according to the present invention;

FIG. 13 is a schematic view of an embodiment of a repetitive microjet device having a phase change mechanism according to the present invention;

FIG. 14 is a schematic view of an embodiment of a repetitive microjet device having an array of phase change mechanism microjets according to the present invention;

FIG. 15 is a schematic view of an embodiment of a repetitive microjet device having an array of microjets actuated by a phase change mechanism according to the present invention;

FIG. 16 is a schematic view of an embodiment of a repetitive microjet device having an electromagnetic microjet according to the present invention;

FIG. 17 is a schematic view of an embodiment of a repetitive microjet device having a spring microjet mechanism according to the present invention;

FIG. 18 is a schematic view of an embodiment of a nozzle of the repetitive microjet device according to the present invention;

FIG. 19 is a schematic view of a further embodiment of a nozzle of the repetitive microjet device according to the present invention;

FIG. 20 is a schematic view of yet another embodiment of a nozzle of the repetitive microjet device according to the present invention;

FIG. 21 is a schematic view of a further embodiment of a nozzle of the repetitive microjet device according to the present invention;

FIG. 22A is a schematic view of the nozzle of the repetitive microjet device shown in FIG. 21;

FIG. 22B is a schematic view of yet another embodiment of a nozzle of the repetitive microjet device according to the present invention;

FIG. 23 is a schematic view of an embodiment of a microprocessor of the repetitive microjet device according to the present invention;

FIG. 24 is a schematic view of another embodiment of a repetitive microjet device according to the present invention;

FIG. 25 is a schematic view of still a further embodiment of a repetitive microjet device according to the present invention;

FIG. 26 is a three dimensional view of an embodiment of the component layers of the repetitive microjet device according to the present invention; and

FIG. 27 is a flow chart of an embodiment of a method of using the transdermal microjet device of the present invention.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to those embodiments. On the contrary, the invention is intended to cover alternatives, modifications, and equivalents, which may be included within the spirit and scope of the invention as defined by the appended claims.

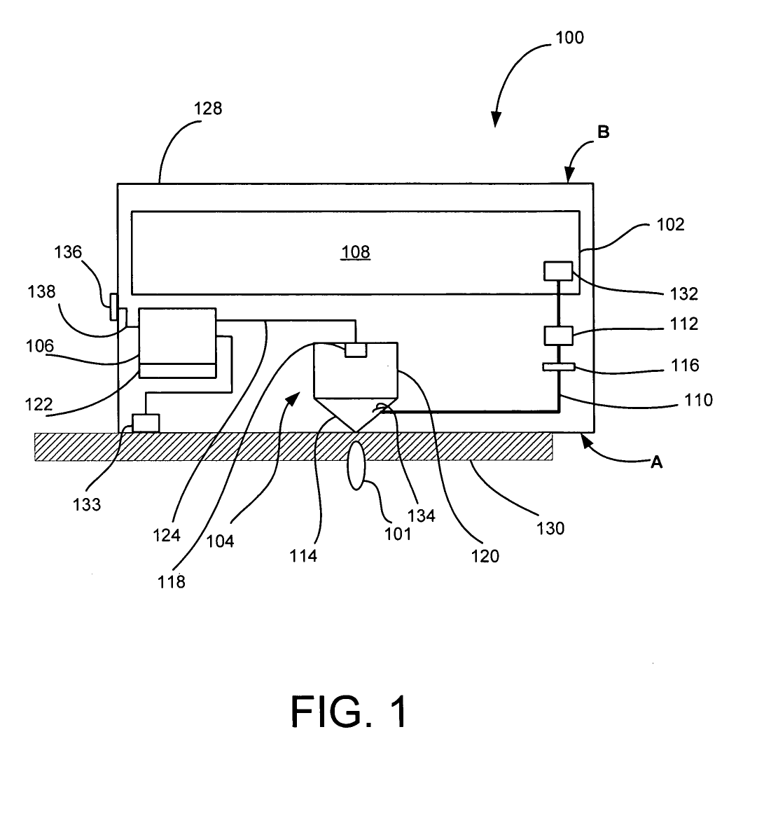

Referring now to a repetitive microjet device 100 as shown in FIG. 1, a drug reservoir 102 is in fluid communication with a microjet 104 that is controlled by a microprocessor 106. Microprocessor 106 is programmable to activate microjet 104 to propel a jet 101 of a substance from microjet 104 towards a biological barrier 130. For ease of reference, surface A of repetitive microjet device 100 is the surface of repetitive microjet device 100 that is positioned toward or adjacent biological barrier 130 and surface B is positioned furthest from biological barrier 130. This orientation will remain consistent throughout the specification and used periodically for orienting the reader.

Furthermore, the repetitive microjet device 100 is capable of repeatable activation. For the sake of clarity, repeatable activation is defined to mean multiple, sequential activation without the need to remove, recharge, or otherwise replenish the device between activation cycles and deactivation cycles. For example, a particular drug administration regime may be to deliver a particular quantity of the drug on each hour for five days. In this example, the repetitive microjet device would activate the force generation mechanism, described below, repetitively, to inject as many micro injections as needed to deliver the prescribed quantity of drug at the first hour. Upon completion of a first hours administration, the device would wait until the next hour, then administer the prescribed quantity of drug a second time. The device would then continue in this manner for the entire five day period. Moreover, according to an embodiment, microprocessor 106 is a simple electronic component or control unit that generates a signal according to predetermined or preprogrammed timing. The timing of the signal can be sequential, but is not limited to sequential timing. The signal then generated by the control unit activates the microjet to propel a jet of fluid toward the biological barrier.

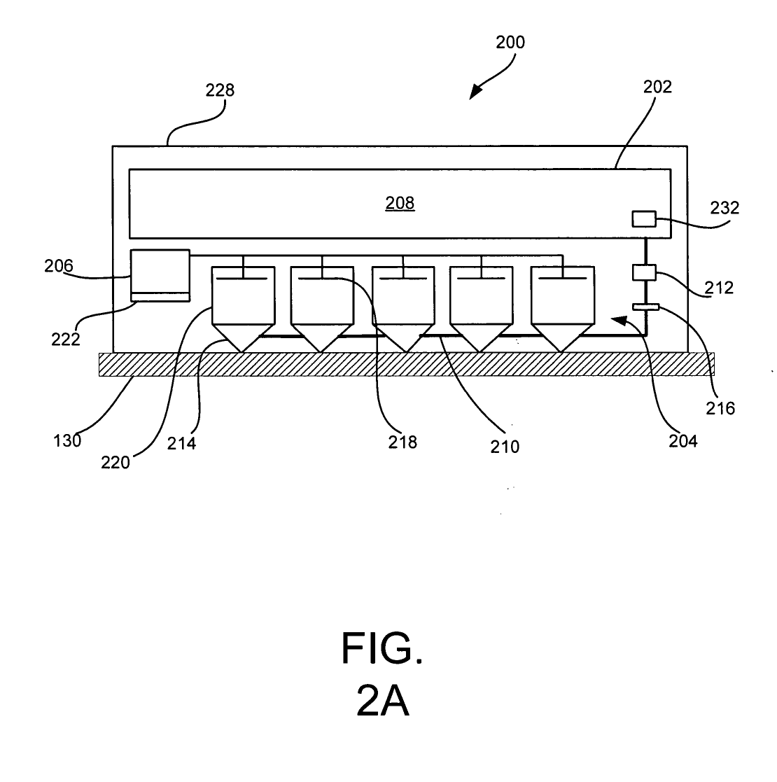

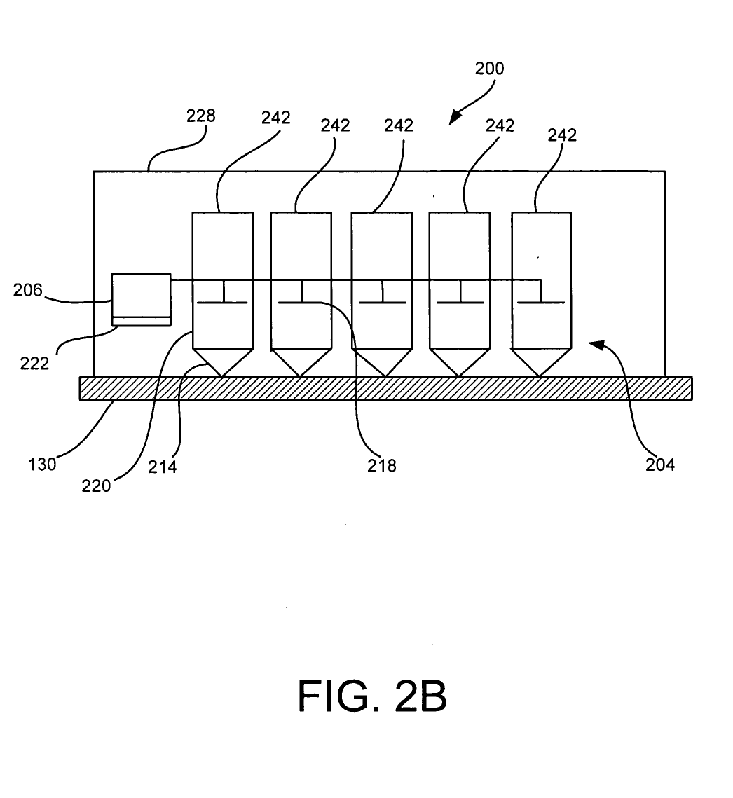

According to another embodiment, as shown in FIGS. 2A and 2B, repetitive microjet device 200 includes a microprocessor 206 that controls an array of microjets 204. The array of microjets 204 can deliver a larger quantity of a substance across a larger surface area of biological barrier than single microjet 104 of FIG. 1. Furthermore, the array of microjets 204 can deliver multiple substances and/or deliver substances in a pattern to optimize administration of a particular substance through biological barrier 130. Preferably the transdermal microjet device, as shown in FIGS. 1, 2A and 2B, provides a sustained period of substance delivery that is not less than about 500 ms and not more than about 10 days.

For simplicity and clarity the following description will primarily describe in detail the components of the single transdermal microjet device 100, as shown in FIG. 1. Reference will be made to the array embodiment, such as that shown in FIGS. 2A and 2B, however, it should be appreciated that the description of the components is equally applicable to each embodiment and not limited to an embodiment utilizing a single microjet.

Transdermal microjet device 100 includes a housing 128. Housing 128 can be constructed from a plastic, metal, ceramic, or other suitably bio-compatible material. Preferably, housing 128 is constructed from a polymer based material such that transdermal microjet device 100 is semi-flexible, can conform to the contour of a surface to which it is applied, is biocompatible, and is drug inert. For example, if transdermal microjet device 100 is configured as a drug delivery patch, it would be advantageous that housing 128 can flex to conform to the contour of the human body at the position at which it is applied. Furthermore, it would be advantageous for transdermal microjet device 100 to be disposable and have a low manufacturing cost. However, it is perceived that it may be advantageous to construct transdermal microjet device 100 from a material not a polymer such that, for example, transdermal microjet device 100 can be sterilized and reused. It may be further preferable to construct transdermal microjet device 100 such that the components are not contained within a single housing. According to such an embodiment, the microprocessor may be separate from the reservoir, which can both be separate from the delivery portion configured to interface with a biological tissue. In such an embodiment, the components; microprocessor, reservoir, and delivery portion are in fluid, electrical, or both communication with each other.

Reservoir 102, as shown in FIG. 1, is configured to house a substance to be ejected from microjet 104. Hereinafter, the substance housed in reservoir 102 and ejected from the microjets will be referred to as injectate 108. Typically injectate 108 is in a liquid form at the time of injection and can be a drug composition, saline solution, emulsion of drug in fluid media, suspension of drug in fluid media, drug coated liposomes in fluid media, drug or drug coated particulates in fluid media, or the like.

According to a preferred embodiment, reservoir 102 can be pressurized such that injectate 108 contained within is urged out of reservoir 102. Alternatively, injectate 108 can be actively pumped out of reservoir 102 by a pump 132.

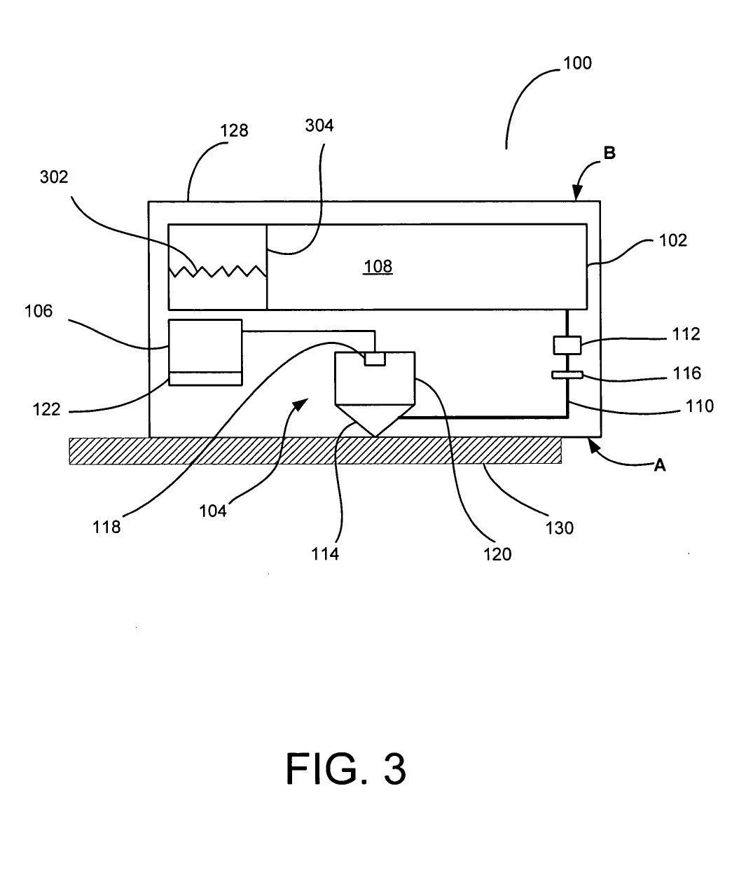

According to an embodiment, as shown in FIG. 3, pressurization of injectate 108 in reservoir 102 can be generated from a spring 302 applying a compressive force to a plunger 304. Spring 302 can be positioned with one end against an inside wall of reservoir 102 and the other end against plunger 304. When reservoir 102 has a full volume of injectate 108, spring 302 is compressed such that spring 302 exerts a pressure against plunger 304. As injectate 108 is expelled during use of transdermal microjet device 100, described in detail below, and the volume of injectate 108 reduces within reservoir 102, spring 302 causes plunger 304 to move, thereby reducing the working volume of reservoir 102. Thus, injectate 108 remains under pressurized conditions and is urged from reservoir 102. It will be appreciated by one of ordinary skilled in the art that the size and rate of spring 302 can be chosen to satisfy the conditions of a particular reservoir volume, density of injectate, viscosity of injectate, or the like to produce a desired pressure at all volumes of injectate 108 in reservoir 102. Alternatively, pressurization of reservoir 102 can be accomplished through a high pressure gas configured to drive plunger 304. Accordingly, the high pressure gas provides the force to move the plunger, thereby reducing the working volume of reservoir 102 maintaining injectate 108 under sufficient pressure.

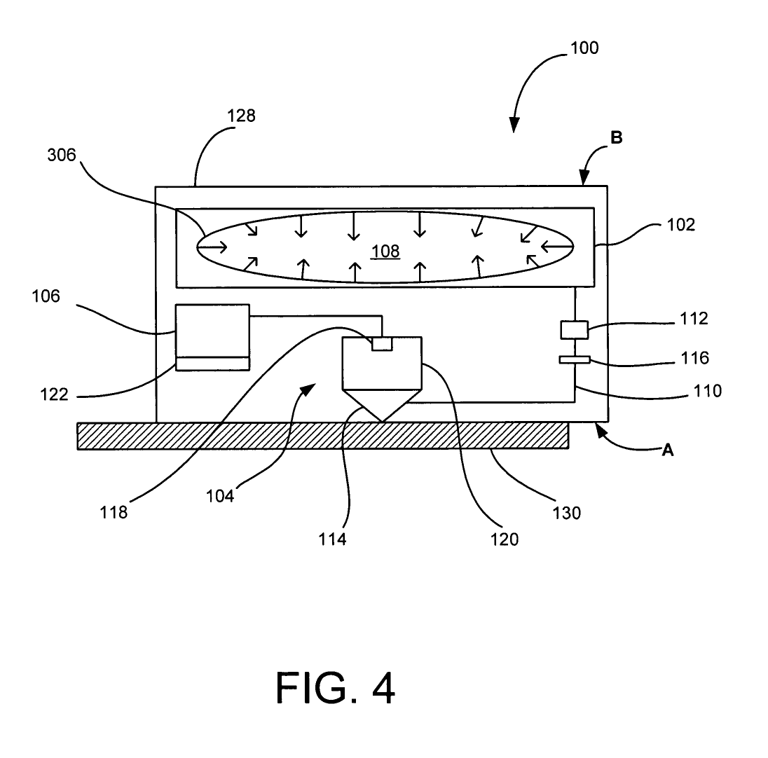

In yet another embodiment, as shown in FIG. 4, reservoir 102 can house a balloon type bladder 306 that is constructed from an expandable elastomeric type material. The balloon type bladder 306 expands when filled with injectate 108. The expanded balloon type bladder 306 thereby renders a force, in the direction of the arrows, urging the injectate 108 from within the bladder 306. Alternatively, the reservoir 102 can itself be constructed from an elastomer type material which expands when filled and produces a force urging the contents of reservoir 102 out of the reservoir.

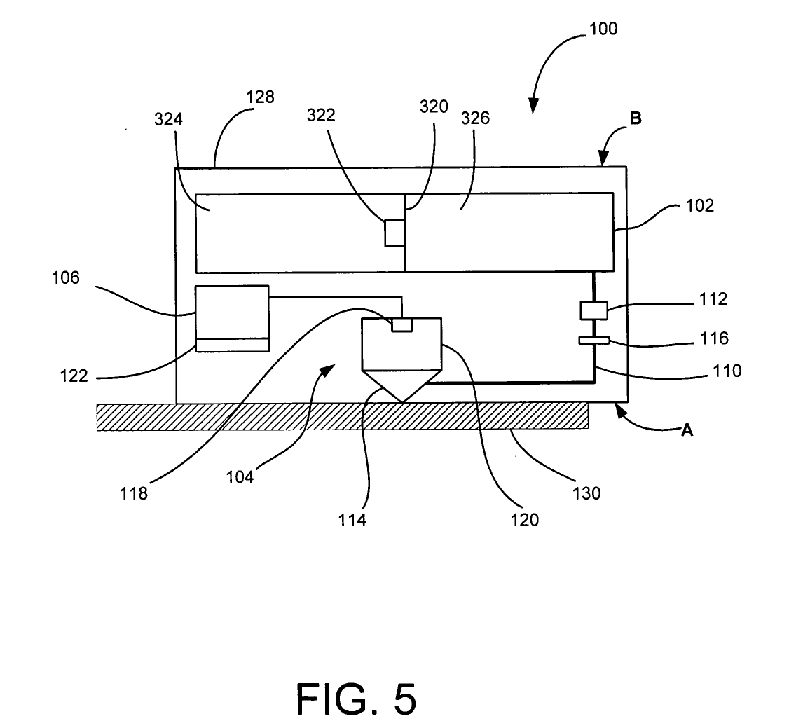

In a preferred embodiment, reservoir 102 can be divided into more than one internal chambers as shown in FIG. 5. In many instances drug components have a longer shelf life if stored in a dry powered form or other form. Therefore, it can be advantageous to maintain the components of reservoir 102 in separate compartments. Accordingly, reservoir divider 320, FIG. 5 divides reservoir into two or more separate chambers 324 and 326. Therefore, two or more injectate components can be discretely maintained. FIG. 5 shows two chambers but it will be appreciated by one of ordinary skill in the art that reservoir 102 can be divided into many chambers having equal volumes or many chambers having different volumes each of which can be combined at one time or at separate times forming multiple stages of injectate for administration at different administration intervals.

According to a preferred embodiment, reservoir 102 has a volume that is not less than about 100 μl and not greater than about 500 ml. In an alternative embodiment, it is preferred that the volume of reservoir 102 is not less than about 150 μl and not greater than about 1 ml. In yet another embodiment, it is preferred that the volume of reservoir 102 is not less than about 200 μl and not greater than about 750 μl.

Reservoir divider 320 is configured to be ruptured my a rupture mechanism 322 prior to administration such that the compositions housed with the divided reservoir can mix in preparation for administration. Preferrably reservoir divider 320 is constructed from biocompatible polymeric foils such as polyethylene, polystyrene, polyethyleneterephthalate (PET), and elastomeric polymers such as polydimethylsiloxane (PDMS), however, it will be appreciated by one of ordinary skill in that art that any thin, non-permeable, drug inert membrane is a candidate for dividing the reservoir into multiple compartments.

Rupture mechanism 322 can be, for example, a ball placed in one of the reservoir chambers. In use, upon shaking or manipulation of the repetitive microjet device 100, the ball moves separately from the device and impacts reservoir divider 320, thereby rupturing reservoir divider 320 and allowing mixing of the different compositions housed in the reservoir chambers 324 and 326. Along with rupturing reservoir divider 320, the ball can facilitate mixing of the drug compositions, thereby, ensuring proper mixing of the injectate prior to administration.

According to an alternative embodiment, rupture mechanism 320 can be a mechanism that is controlled by microprocessor 106. This rupture mechanism 320 can be, for example, a piezoelectric mechanism. According to such an embodiment, the microprocessor 106 controls the delivery of a supply of voltage, from the power supply to the piezoelectric rupture mechanism. The piezoelectric rupture mechanism creates mechanical pressure waves such as ultrasound waves in the fluid media upon application of an alternating current. These mechanical pressure waves serve to rupture the reservoir divider.

According to such an embodiment, the reservoir can be divided into multiple reservoirs. Microprocessor 106 can control the timing and sequence of rupture of reservoir dividers 320 such that particular reservoir dividers can be ruptured, thereby releasing compositions for mixing. In this manner, only the portion of compositions that will be currently administered, i.e., a current dosage, is mixed and the remaining quantity of composition remains in stable discrete form in discrete reservoirs. As a result, repetitive microjet device 100 can discretely house treatment compounds, which can remain viable for long periods of time for repetitive delivery of treatments over sustained periods.

Microprocessor controlled rupture mechanism 320 can be, for example, an electrical impulse generated by microprocessor 106. Each independent reservoir divider 320 can include electrodes that, when activated, cause the respective reservoir divider to rupture, thereby allowing subsequent mixing of compositions for administration. Alternatively, rupture mechanism 320 can be, for example, physical disruption of reservoir divider 320 such as by spearing, twisting, shock-wave, explosion, or the like. Rupture mechanism may be any such mechanism that is capable of rupturing or disrupting the integrity of the non-permeable reservoir dividers.

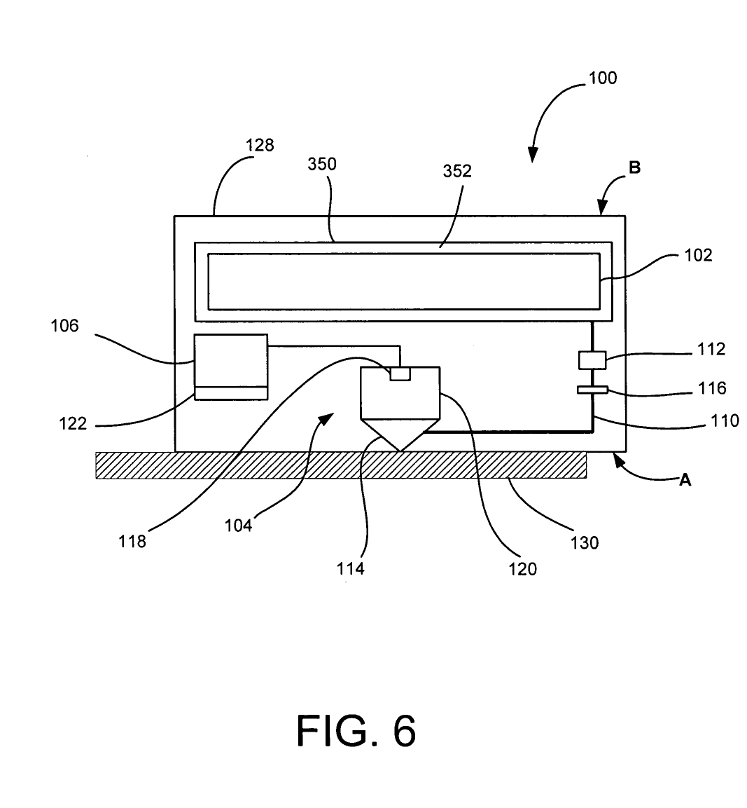

Often in medical applications the treatment of patients requires drugs which may be illicit outside of the prescription care of a physician. Some of these drugs may be addictive and eagerly sought by individuals for use outside of the prescribed use. Because transdermal microjet device 100 includes reservoir 102 that may store a quantity of such drug components for repetitive and sustained administration, it is conceivable that some individuals may seek to extract the drug components from reservoir 102 for illicit uses. Therefore, it can be advantageous to include an antagonist reservoir 350 in transdermal microjet device 100, as shown in FIG. 6. Antagonist reservoir 350 is associated with reservoir 102 and preferably contains an antagonist 352 to the drug component or components, i.e., injectate 108, contained in reservoir 102.

Antagonist reservoir 350 is designed to be easily disrupted, releasing antagonist 352 from within when the transdermal microjet device 100 is manipulated or tampered with in a manner sufficient to extract injectate 108 from reservoir 102. When antagonist reservoir 350 is disrupted the antagonist 352 will be released, such that the injectate 108 drug components will be inactivated.

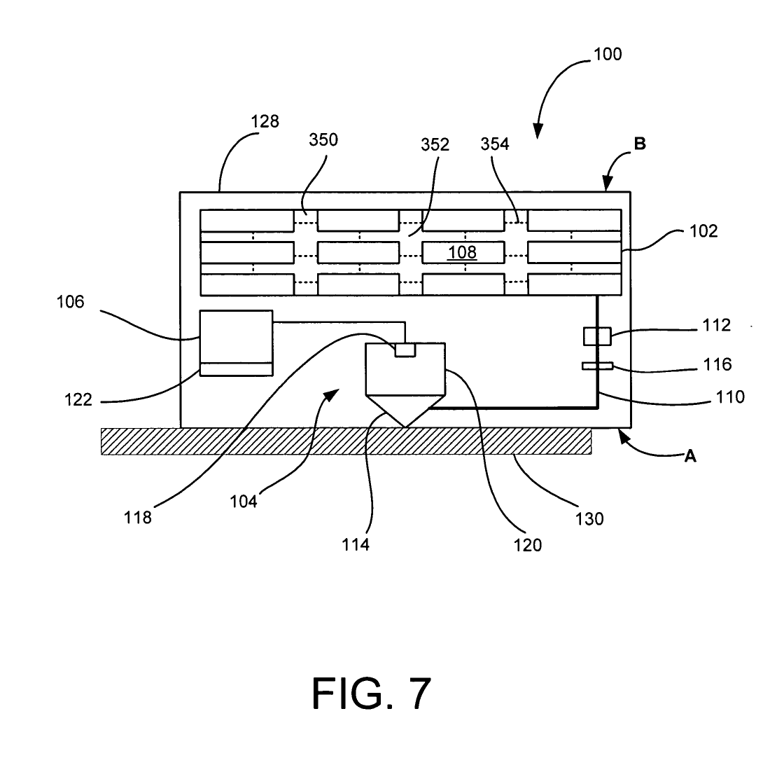

Antagonist reservoir 350 can be, for example, a reservoir that is positioned to surround reservoir 102 and constructed from a material which will be disrupted more easily than reservoir 102. Alternatively, as shown in FIG. 7, antagonist reservoir 350 can be, for example, a grid like pouch structure throughout reservoir 102 and configured with break zones 354 such that the break zones 354 will break in response to physical manipulation prior to reservoir 102 breaking, thereby releasing antagonist into injectate 108 and rendering injectate 108 ineffective.

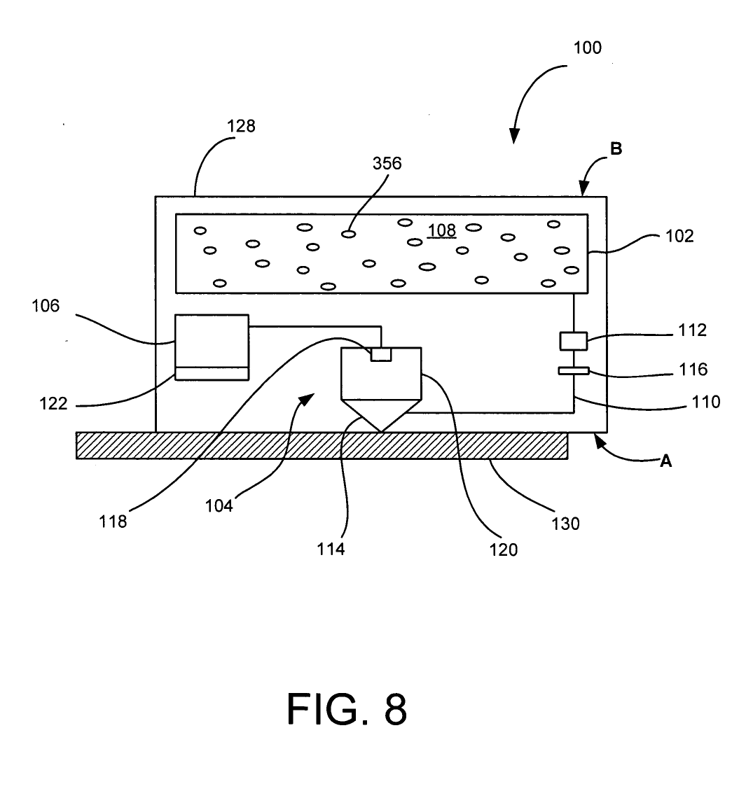

In yet another embodiment, as shown in FIG. 8, antagonist reservoir 350 can be, for example, multiple microspheres 356. Multiple microspheres 356 are preferably constructed to rupture, thereby releasing an antagonist, upon excessive manipulation of reservoir 102.

Referring back to FIG. 1, reservoir 102 is in fluid communication with microjet 104 through feed line 110. Feed line 110 can be a tube, chamber, groove in laminates of housing 128 (described in detail below), such that, when laminates are assembled a corridor is formed between reservoir 102 and microjet 104, or another configuration forming a mechanism that allows injectate 108 to transfer between reservoir 102 and microjet 104.

Feed line 110 can include a valve 112. Valve 112 is preferably a one-way valve such that flow of injectate 108 is restricted to flowing in the direction toward microjet 104, and restricted from flowing in the reverse direction, toward reservoir 102. Feed line 110 extends to and is fluidly coupled with nozzle 114 of microjet 104.

In a preferred embodiment, feed line 110 contains a pressure regulator 116 to regulate the pressure in feed line 110. Injectate 108 can be maintained under pressure in reservoir 102, as described above, to a higher pressure than the desired pressure in nozzle 114. Therefore, pressure regulator 116 functions to regulate downstream pressure in feed line 110 such that the pressure of injectate 108 at nozzle 114 is maintained at an appropriate level. The appropriate level will be appreciated by one of ordinary skill in the art to be a pressure that fills nozzle 114 with injectate 108 but does not overcome the forces that maintain injectate 108 within nozzle 114, as described in more detail with respect to the description of nozzle 114 herein.

Microjet 104, FIG. 1, will now be described, however, it will be appreciated that the description is equally applicable to the microjets 204 of an array embodiment such as that shown in FIGS. 2A and 2B. Microjet 104 generally includes a force generating mechanism 118, a chamber 120, and a nozzle 114.

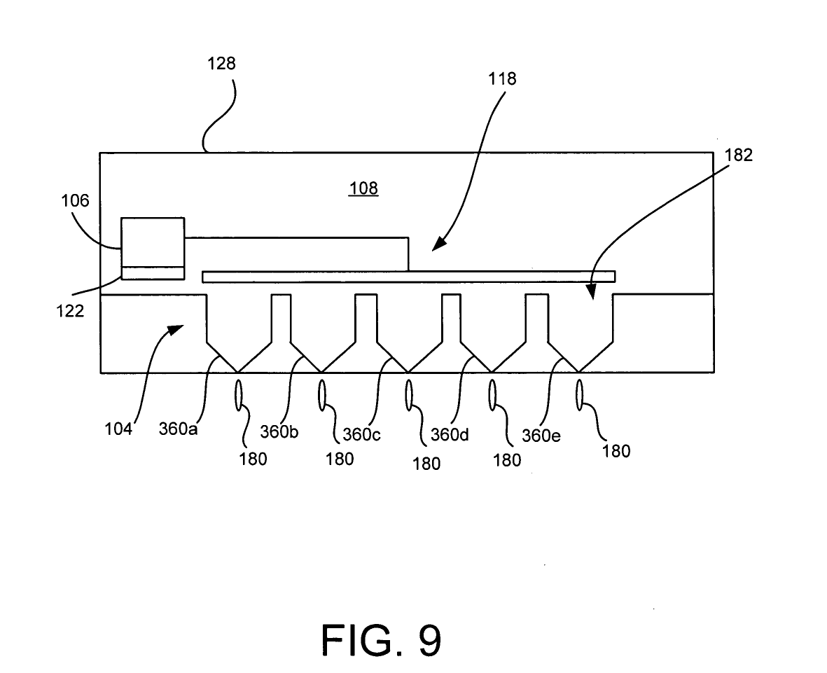

Force generation mechanism 118, FIG. 1, is generally positioned within repetitive microjet device 100 such that force generated from mechanism 118 is directed toward side A of repetitive microjet device 100. Generally, each microjet 104 can include a discrete force generation mechanism 118, as shown in FIG. 1. Alternatively, a group of microjets 360a-360e can be actuated by one force generation mechanism 118, as shown in FIG. 9. In use, force generation mechanism 118 generally functions to change the pressure within chamber 120, thereby, accelerating injectate 108 within the chamber 120 toward nozzle 114. Following activation of the force generation mechanism, the accelerated injectate becomes ejected from each nozzle 114, producing a jet of injectate ejected therefrom. In a preferred embodiment, the jet of injectate contains not less that about 1 pl and not more than about 800 nl of injectate. In a more preferred embodiment, the jet of injectate contains not less than about 100 pl and not more than about 1 nl of injectate. According to a preferred embodiment, the force generation mechanism generates a pulse width or pressure change within the chamber at a rate of not less than about 5 ns and not more than about 10 μs. In an alternative embodiment the pulse width is not less than about 0.5 μs and not more than about 5 μs. In yet another alternative embodiment, the pulse width is not less than about 1 μs and not more than about 3 μs. In a preferred embodiment, the force generation mechanism generates not more than about 100 pulses per second. In a more preferred embodiment, the force generation mechanism generates not less than about 5 pulses per second and not more than about 15 pulses per second.

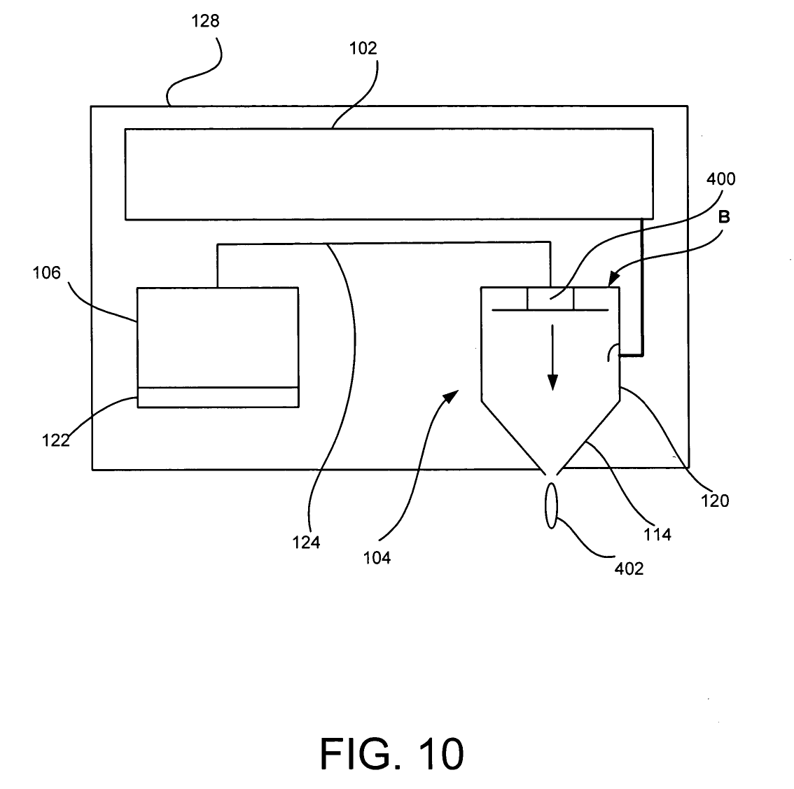

According to an embodiment, force generation mechanism 118 is a piezoelectric mechanism 400, as shown in FIG. 10. A piezoelectric is a dielectric crystal that generates a voltage when mechanical stress is applied to the crystal or, on the other hand, mechanically stresses when a voltage is applied to the crystal. Piezoelectric devices are well known and the operation of a piezoelectric will be apparent to one of ordinary skill in the art. Piezoelectric mechanism 400 is positioned against distal side B of microjet 104. Distal wall B of microjet 104 is constructed to withstand the mechanical force generated by piezoelectric mechanism 400 such that the wall does not flex when piezoelectric mechanism 400 mechanically stresses. As a result, the mechanical stress or deformation of piezoelectric mechanism 400 is concentrated in proximal direction A, toward nozzle 114. Piezoelectric mechanism 400 is configured to act as a plunger, creating a pressure change within injectate 108 in the proximal direction during mechanical deformation, thereby generating a jet of injectate 402 ejected from nozzle 114.

Microprocessor 106, described in more detail below, is connected to piezoelectric mechanism 400 through circuitry 124, FIG. 10. In use, when an administration of injectate 108 is scheduled or demanded, as described in more detail below, microprocessor 106 controls the supply of an electric voltage, stored in power supply 122, to piezoelectric mechanism 400. In response to the electric voltage, piezoelectric mechanism 400 mechanically stressed to deform and generate the pressure change in chamber 120 (FIG. 1).

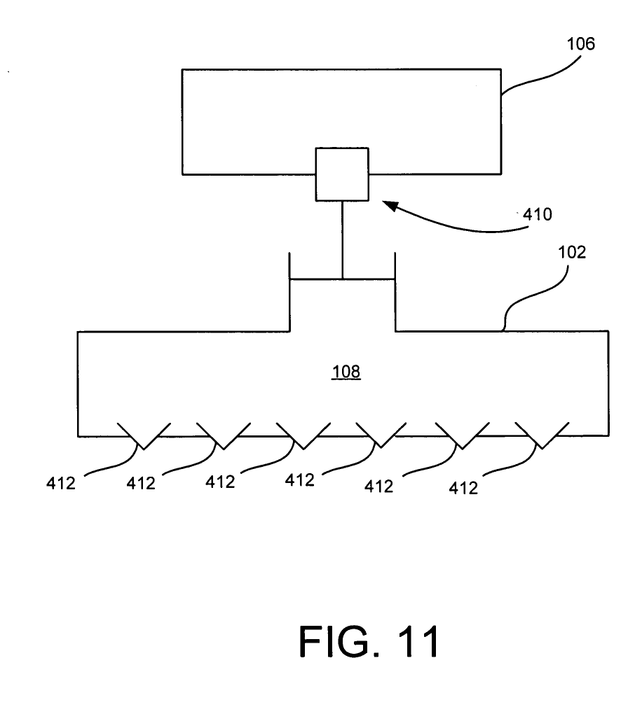

According to the embodiment shown in FIG. 11, one piezoelectric mechanism 410 can actuate multiple nozzles 412. During administration of injectate 108 from reservoir 102, the volume of injectate 108 decreases. In response to the decrease in volume, the voltage applied to piezoelectric mechanism 410 is increased, such that a larger physical deformation of piezoelectric mechanism 410 is generated. The larger physical deformation of piezoelectric mechanism 410 is correlated to the decrease in volume of injectate in reservoir 102, such that the relative same pressure change is generated within reservoir 102, resulting in a consistent ejection force of injectate from nozzle 114 for consistent and predictable application and delivery of injectate.

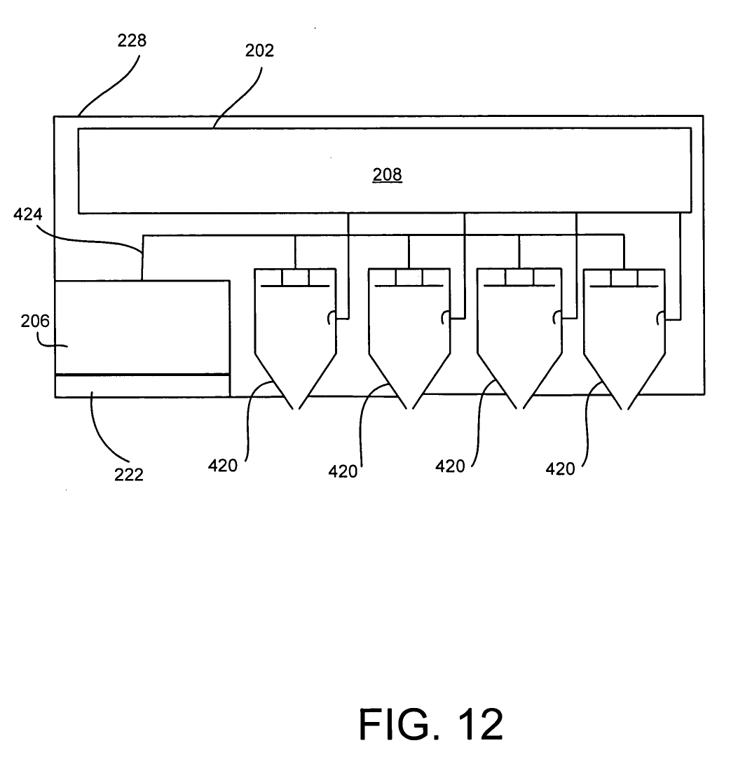

According to an embodiment having an array of piezoelectric microjets 420, FIG. 12, circuitry 424 can independently couple to each microjet 420. Therefore, microprocessor 206 can discretely control the timing and sequence of deformation of each piezoelectric mechanism 420. As a result, the pattern of administration of injectate 208 can be controlled for optimized administration results depending on the type of injectate that is required, e.g., insulin for treating diabetes. The administration pattern can be varied to optimize absorption and/or diffusion into the systemic circulatory system, minimize biological barrier irritation, tailored to a particular patient, or the like, such that patient compliance, drug efficiency, and effectiveness are optimized.

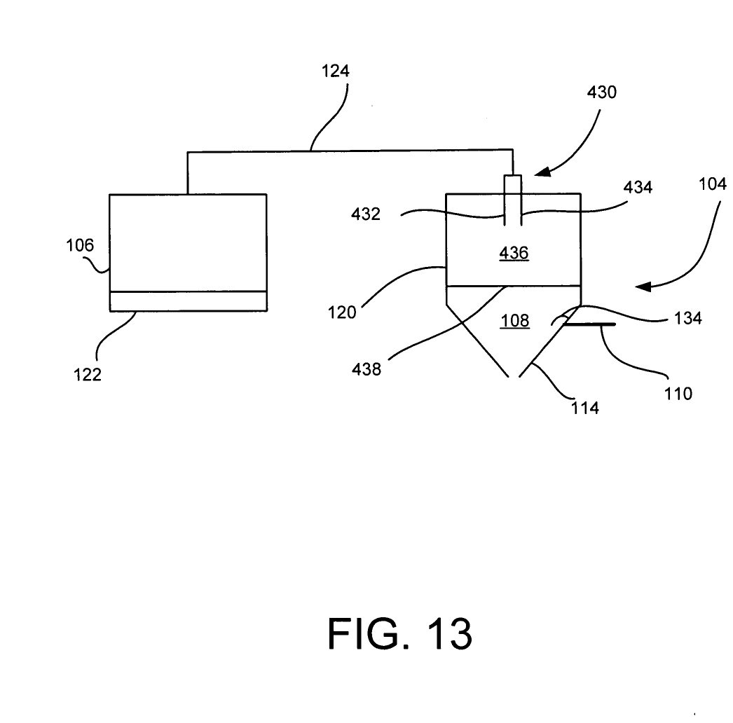

According to an alternative embodiment, the force generation mechanism can be a phase change mechanism 430, as shown in FIG. 13. Phase change mechanism 430 includes two electrodes 432 and 434. Electrodes 432 and 434 penetrate the distal end of microjet 104 and protrude into chamber 120. Chamber 120 is a fully enclosed chamber that houses actuation fluid 436. The distal and lateral sides of chamber 120 are configured to withstand a force generated by phase change mechanism 430, whereas the proximal end of chamber 120 is a flexible membrane 438. Flexible membrane 438 is preferably non-permeable to actuation fluid 436 in chamber 120 and injectate 108 contained in nozzle 114, such that the two compositions do not mix.

Actuation fluid 436 is a fluid that is easily broken down and vaporizes rapidly upon the build-up of a difference in electric charge on electrodes 432 and 434. The actuation fluid 436 is typically a conductive ionic fluid including but not limited to a saline fluid, other salt solutions in water such as aqueous metal halides, i.e., potassium chloride, calcium chloride, and the like, can also be utilized. Furthermore, dielectric materials with low boiling points can also be utilized as actuation fluid 436, such as fluorocarbons.

According to an alternative embodiment, the actuation fluid 436 can be the injectate. Accordingly, the flexible membrane 438 may not be necessary as the entire chamber 120 and nozzle 114 are filled with the fluid that is ultimately injected following activation of the phase change mechanism.

Because the volume of a given amount of fluid increases vastly when the fluid is changed into its gaseous form, generating a vaporization of a given amount of fluid in a fixed volume chamber will vastly increases the pressure with the chamber. Thereafter, the flexible membrane 438 is deformed in the proximal direction, thereby decreasing the volume of nozzle 114. As a result, the injectate 108 is forced in the proximal direction and becomes ejected from nozzle 114, as described in more detail below.

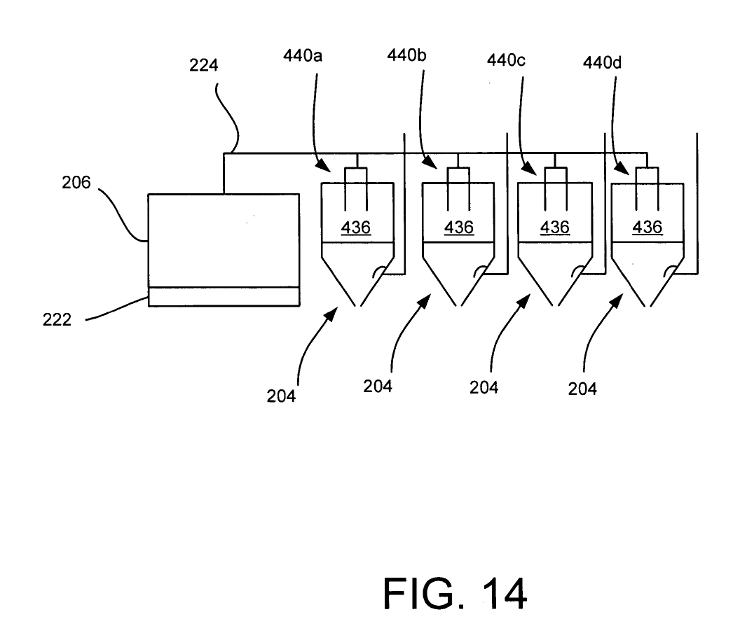

Microprocessor 106 is in electrical communication with phase change mechanism 430 through circuitry 124. Similar to activation of the piezoelectric mechanism, as described above, microprocessor 106 can control the actuation of phase change mechanism 430. Following vaporization of actuation fluid 436, the actuation fluid 436 reforms as fluid and is capable of a repetitive vaporization, thereby, generating a repetitive microjet. In an embodiment employing an array of microjets 204, FIG. 14, microprocessor 206 controls the timing and sequence of firing of phase change mechanisms 440a-440d. By way of example but not limitation, a phase change mechanism can be a pair of electrodes immersed in a saline solution filled nozzle. According to this example, a stainless steel syringe needle, approximately 1 mm in diameter can form the ground electrode and a tungsten wire, approximately 25 μm in diameter can form the positive electrode. A glass cap with an approximately 30 μm diameter opening at one end can form the nozzle. This glass cap caps the syringe-wire electrode pair such that the electrode pair is immersed in the saline solution of the glass cap. Thereafter, when a charge differential is applied to the positive-negative electrode pair, the saline solution is broken down and undergoes a phase change, generating a pressure change within the nozzle/cap.

In an alternative embodiment, actuation fluid 436, FIG. 13, can be maintained separate from injectate 108 by chemical and physical properties of the actuation fluid, therefore, no membrane is required. Accordingly, the actuation fluid can be immiscible with injectate 108 such that the two fluids do not mix. Therefore, a flexible membrane is not required.

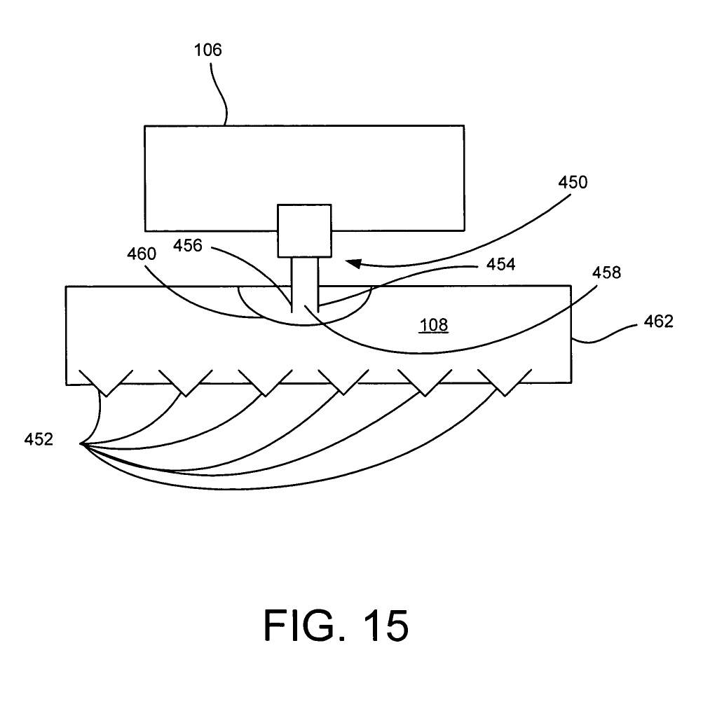

According to another embodiment, as shown in FIG. 15, a single phase change mechanism 450 can actuate multiple nozzles 452. The phase change mechanism 450 includes at least two electrodes 454 and 456. Surrounding electrodes 454 and 456 is actuation fluid 458 housed with a closed volume by flexible membrane 460. During administration of injectate 108, the volume of injectate 108 within reservoir 462 is reduced. Therefore, to generate a constant ejection force of injectate from nozzles 452, a corresponding larger and larger force is generated by phase change mechanism 450, which displaces flexible membrane 460 further and further. Accordingly, multiple nozzles 452 can be actuated by one phase change mechanism 450 with a repetitive ejection force of injectate regardless of the amount of injectate contained in reservoir 462.

The phase change mechanism of the present invention generally operates on a high voltage of not less than about 500V and not more than about 10 kV. The phase change mechanism preferably operates on a voltage of not less than about 1 kV and not more than about 6 kV. In an alternative embodiment, the phase change mechanism of the present invention operates on a voltage of not less than about 3 kV and not more than about 6 kV. The voltage is pulsed at not less than about 5 ns and not more than about lops. In an alternative embodiment the voltage is pulsed at not less than about 0.5 μs and not more than about 5 μs. In yet another alternative embodiment, the voltage is pulsed at not less than about 1 μs and not more than about 3 μs.

The flexible membranes 438 and 460 are preferably constructed from a low Young's modulus elastomer material, such as polydimethylsiloxane (silicone rubber), fluoropolymer (Kalrez), or the like. The preferably thickness of flexible membranes 438 and 460 are not less than about 0.1 μm and not more than about 100 μm. In an alternative embodiment, the flexible membranes 438 and 460 are not less than about 0.5 μm and not more than about 50 μm in thickness. According to yet another embodiment, the flexible membranes 438 and 460 are not less than about 1 μm and not more than about 10 μm in thickness.

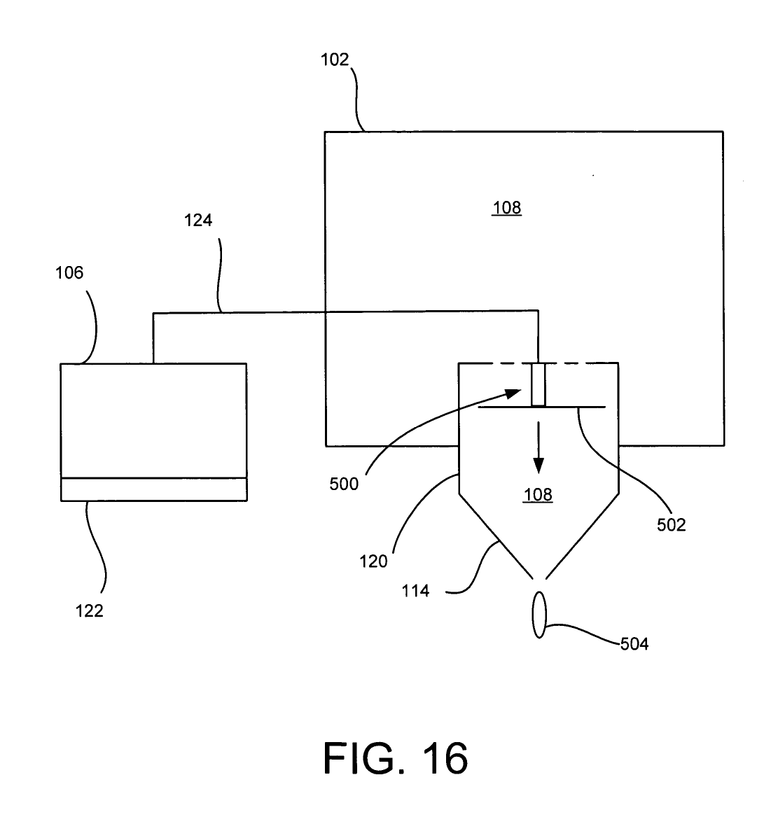

According to yet another embodiment, force generation mechanism 118, (FIG. 1) can be an electromagnetic actuation mechanism 500, such as a solenoid, as shown in FIG. 16. Electromagnet actuation mechanism 500 functions in response to communication from microprocessor 106, descried below, such that a plunger 502 is moved in the proximal A direction, shown by the arrow, in chamber 120 generating movement of injectate 108 and a jet ejection 504 of injectate 108 for administration to a patient. Electromagnet actuation mechanism 500 will be appreciated by one of ordinary skill in the art and, therefore, not described in detail any further.

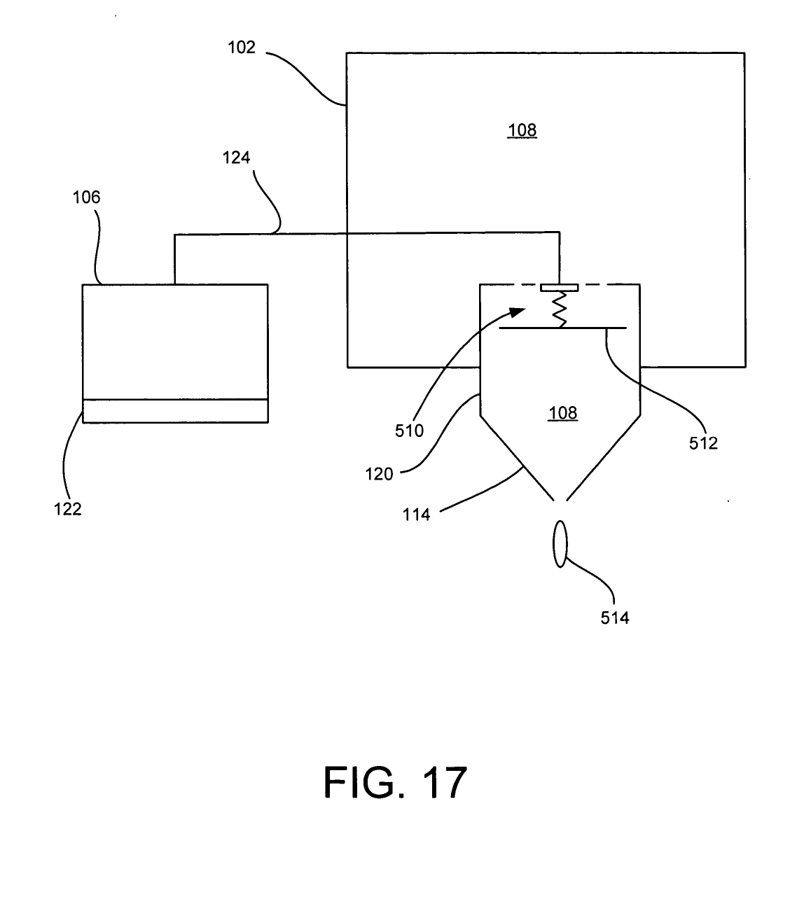

According to yet another embodiment, force generation mechanism 118 (FIG. 1) can be a spring mechanism 510, that operates a plunger 512 as shown in FIG. 17. According to this embodiment, the proximal end of chamber 120 can be open, with no membrane separating the nozzle 114 compartment from chamber 120. Both chamber 120 and nozzle 114 are filled with injectate 108. Therefore, a force generated in injectate 108 of chamber 120 propagates through injectate 108 of nozzle 114 and results in a jet expulsion 514 of injectate 108 from nozzle 114, as described in more detail below.

In still a further embodiment of the present invention, force generation mechanism 118 (FIG. 1) can be a highly pressurized gas which, when activated, moves a plunger and thereby displaces injectate 108 from nozzle 114. According to this embodiment, microprocessor 106 (FIG. 1) controls the movement of the high pressure gas such as to generate a jet of injectate 108 for administration of injectate 108 upon appropriate timing and/or sequence as described above.

In still a further embodiment, the force generation mechanism 118 can be an explosive mechanism. The explosive mechanism can include, for example, a mixture of chemicals that upon the delivery of a voltage or other type of ignition source, excite and produce an explosion. The explosion thereafter generates a pressure change within chamber 120 and drives injectate from nozzle 114 and into the adjacent biological tissue.

Chamber 120, FIG. 1, is preferably constructed from a polydimethylsiloxane, commonly know as PDMS or silicone, however, other polymers, ceramic, or metal materials can also be utilized. The diameter of chamber 120 is not less than about 0.1 μm in diameter and not greater than about 500 μm. More preferably, the diameter of chamber 120 is not less than about 0.5 μm and not greater than about 100 μm. Most preferably, the diameter of chamber 120 is not less than about 1 μm and not greater than about 10 μm.

Referring to FIG. 1, chamber 120 is in fluid communication with nozzle 114 of microjet 104. As force generation mechanism 118 creates a pressure change and/or volume change within chamber 120 and nozzle 114, thereby ejecting injectate 108 from nozzle 114, chamber 120 and nozzle 114 must be re-stocked with injectate 108 so as to be prepared for a subsequent actuation, thereby producing a repetitive microjet. Following actuation of force generating mechanism 118, chamber 120 is refilled with injectate 108 from reservoir 102 during operation of the repetitive microjet device 100.

As described above, an embodiment of the present invention utilizes a feed line 110 maintaining reservoir 102 and nozzle 114 in fluid communication. Also, as described above, reservoir 102 can be pressurized or include a pump 132, such that injectate 108 is urged down feed line 110 and into nozzle 114, thus refilling nozzle 114 and chamber 120 following each ejection of injectate 108. Alternatively, feed line 110 can be coupled with and empty into chamber 120 rather than nozzle 114.

In a preferred embodiment, the diameter of the opening of feed line 110 at the intersection of chamber 120 and/or nozzle 114 is substantially smaller than the opening of nozzle 114 such that flow of injectate 108 into feed line 110 in the reverse direction is negligible. Also, there can be a deflector plate 134, FIG. 1, positioned over the opening of feed line 110 into nozzle 114 positioned to deflect injectate 108 from entering feed line 110 in the reverse direction during actuation of microjet 104. According to another embodiment, valve 112, (FIG. 1) can be positioned at the point where feed line 110 engages nozzle 114 such that injectate 108 does not enter feed line 110 in the reverse direction during actuation of microjet 104.

According to an alternative embodiment, injectate 108 refills nozzle 114 and chamber 120 by capillary action if reservoir 102 is not pressurized.

In an alternative embodiment, FIG. 9, the distal portion of chamber 120 extends into reservoir 102, has openings into reservoir 102, or has a semi-permeable membrane between chamber 120 and reservoir 102. As the force generation mechanism 118 generates a pressure difference in injectate 108 sufficient to eject jets 180 from microjets 104, injectate enters chamber 120 through openings 182 to equalize the pressure within chamber with the pressure in reservoir 102.

According to yet another embodiment, as shown in FIGS. 11 and 15, the reservoir for housing injectate can also function as the chamber.

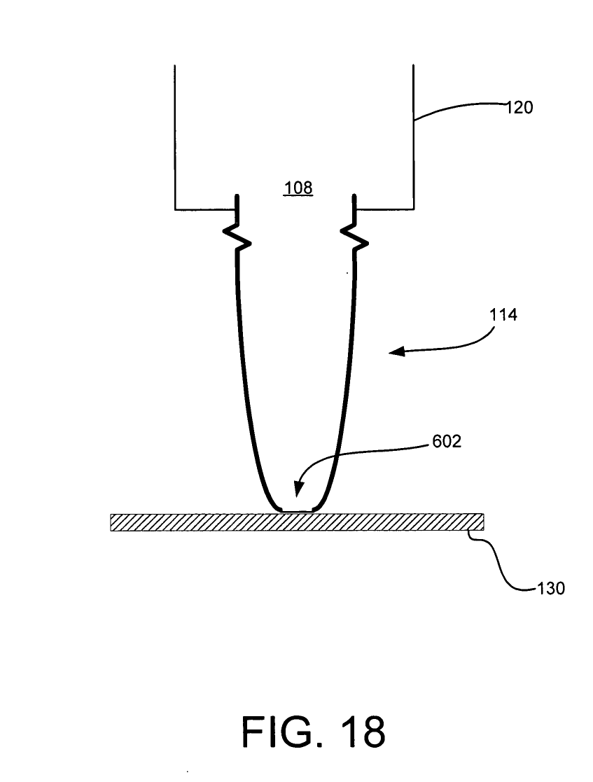

FIG. 18 shows a general configuration of nozzle 114. A distal end of nozzle 114 is coupled with chamber 120 and a proximal end of nozzle 114 is configured to interact with a biological barrier 130. In use, as force generation mechanism 118 (FIG. 1) generates a pressure change in chamber 120, the pressure change causes injectate 108 within chamber 120 and nozzle 114 out of nozzle 114 in a jet form. Nozzle 114 preferably tapers to a smaller cross-sectional diameter toward the proximal opening 602 of nozzle 114. Because the initial volume of injectate 108 that is accelerated is greater than the volume of nozzle 114 as nozzle 114 tapers proximally, injectate 108 must accelerate to a greater velocity. Upon reaching the opening of nozzle 114, the accelerated injectate becomes ejected from nozzle 114 as a jet of fluid. It will be appreciated by one of ordinary skill in the art that the nozzle dimensions, chamber volume, viscosity of injectate, and the like can be altered to configure the ejected jet of injectate to carry a predetermined amount of force such that the jet of injectate will penetrating a biological barrier 130 and deposit the injectate at a desired depth in the adjacent tissue.

According to the nozzle of FIG. 18, nozzle 114 is configured with a semi-blunt proximal end where it can gently abut biological barrier 130. Injectate 108 within nozzle 114 also interfaces with biological barrier 130. Therefore, when force generation mechanism 118 is actuated, an administration quantity of injectate is propagated through injectate 108 in nozzle 114 and forced through the initial layers of the adjacent biological barrier 130.

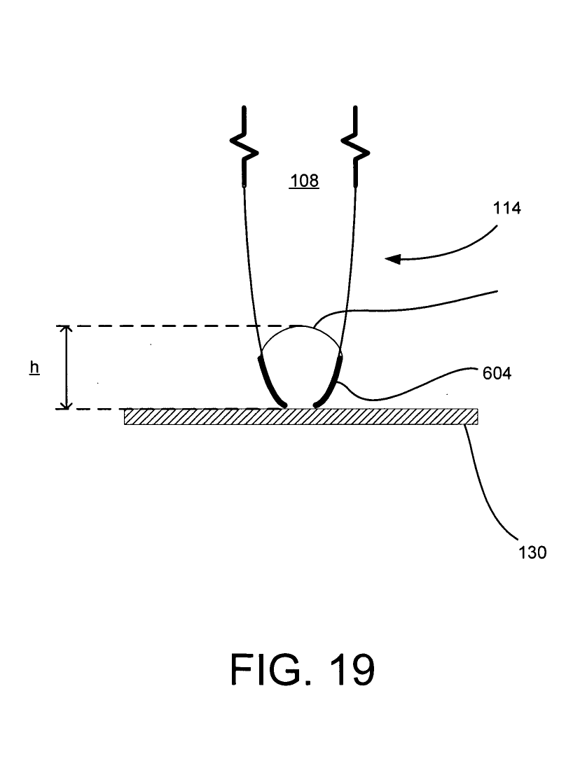

According to an embodiment, as shown in FIG. 19, the proximal end 604 of nozzle 114 can include a coating to make it repel injectate 108, constructed from a composition which repels injectate 108, or the like. For example, if injectate 108 is a hydrophilic substance, the proximal end 604 of nozzle 114 can be coated with or constructed from a hydrophobic substance, thereby repelling injectate 108 from passively entering the proximal end 604 of nozzle 114. In this embodiment, the injectate 108 is retained a set distance, h, from the surface of biological barrier 130 during resting stages of the device. Therefore, if injectate 108 has a tendency to irritate biological barrier 130 or produce another negative effect on biological barrier 130 if left in contact with biological barrier 130, these events will be minimized. Furthermore, in accord with this embodiment, a more accurate quantity of administered injectate 108 can be predicted and delivered because the injectate will not be able to diffuse through biological barrier 130 or enter biological barrier 130 except as the jet propulsion stream during administration.

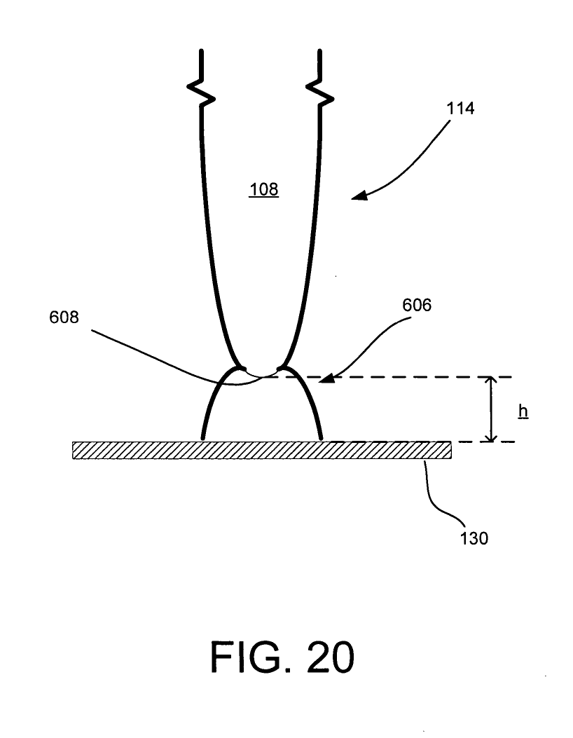

Alternatively, the proximal end of nozzle 114 can have a convergent/divergent configuration 606, as shown in FIG. 20. According to this embodiment, the position of the injectate 108 can be determined to retain the meniscus of the injectate 608 at an optimum distance, h, from biological barrier 130. Height, h, is determined and set as a distance between the meniscus of injectate 608 and biological barrier 130 that allows for penetration of the administered jet of injectate 108 to penetrate biological barrier 130 a set distance. According to an embodiment, the height, h, can be not less than about 0 μm and not more than about 5000 μm from the surface of the biological barrier. According to another exemplary configuration, for example, the stratum corneum is about 10 μm-15 μm in thickness and the epidermis is about 50 μm-100 μm in thickness below the stratum corneum. Therefore, if the epidermis is the target zone for the injected injectate, height, h, can be set at a distance that results in the injected injectate penetrating to a distance of not less than about 10 μm and not more than about 500 μm. In an alternative embodiment the injectate penetrates to a depth of not less than about 25 μm and not more than about 100 μm below the surface of the biological barrier.

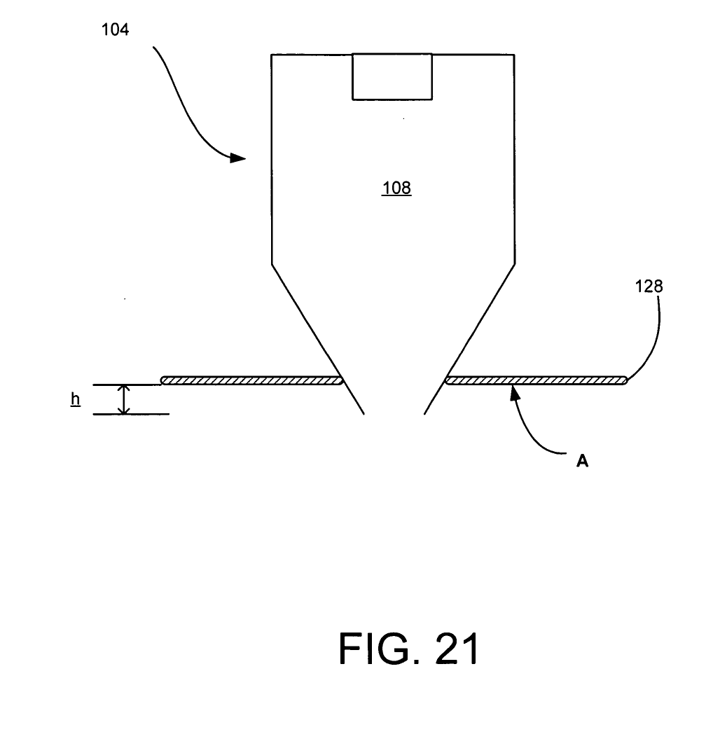

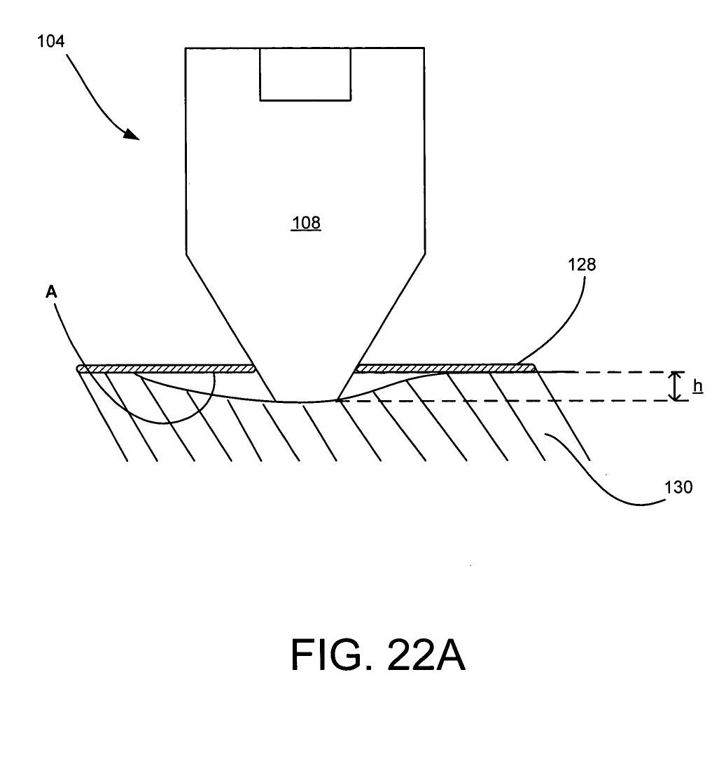

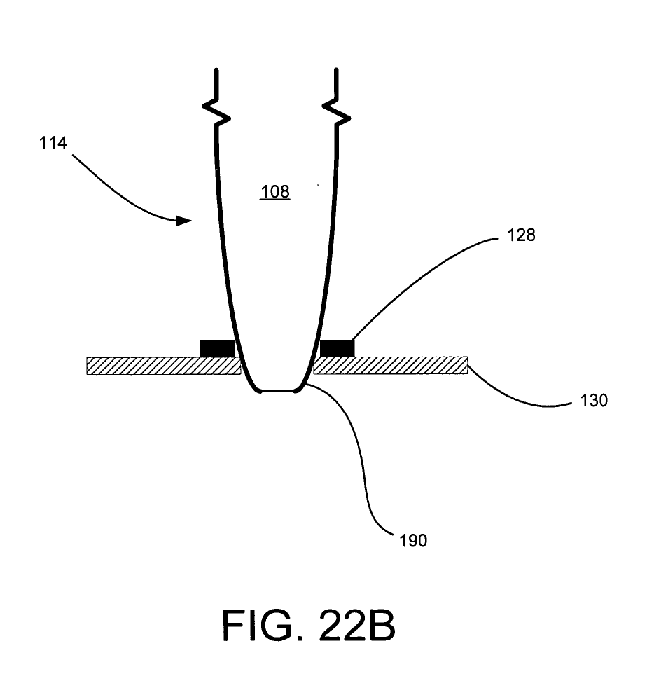

According to an embodiment of the present invention, as shown in FIG. 21, nozzle 114 protrudes a distance, h, from housing 128. During use, proximal surface A of housing 128 can be positioned directly against a biological barrier 130, as shown in FIG. 22A, such that nozzle 114 will automatically be positioned at a preferred orientation with biological barrier 130. Furthermore, the protrusion of nozzle 114 from the proximal surface A of housing 128 applies a tension to biological barrier 130 when the transdermal microjet device 100 is in position against biological barrier 130. Putting biological barrier 130 under tension or pre-load facilitates the ejection jet from microjet 104 to penetrate biological barrier 130. The pre-loading removes or reduces the elasticity properties from biological barrier 130. Therefore, an accurate quantity of injectate that actually penetrates the biological barrier can be calculated and the device can be utilized to deliver precise dosing requirements. As a result, a known and constant contact pressure will be applied between nozzle 114 and biological barrier 130. Thus a user simply need apply proximal side A of housing 128 against biological barrier 130 and nozzle 114 will be properly positioned for optimal administration of injectate.

According to an alternative embodiment, as shown in FIG. 22B, the proximal end of nozzle 114 that protrudes beyond housing 128 can be configured to be positioned into or through the initial layer of biological barrier 130. In use, the first several jet injectates produced from microjet 104 produce a pore 190 through or into biological barrier 130 and the application force of applying transdermal microjet device 100 to the biological barrier 130 results in the positioning of proximal tip of nozzle 114 into the pore 190 generated by the jet injectates. Accordingly, following insertion of the proximal tip of nozzle 114 into or through the biological barrier 130, injectate 108 in nozzle 114 can passively diffuse into biological barrier 130.

Nozzle 114 preferably has an orifice diameter of not less than about 1 μm and not greater than about 500 μm. According to another embodiment, nozzle 114 has an orifice diameter not less than about 25 μm and not greater than about 250 μm. More preferably, nozzle 114 has an orifice diameter not less than about 30 μm and not greater than about 75 μm.

Nozzle 114 can be manufactured by many known methods in the art, for example, one method includes heating a glass tube and pulling the tube to obtain a desired diameter then scribing, braking, and polishing the tube to perfect the nozzle. Another more preferable method includes molding the nozzle or injection molding the nozzle from a master mold. Still another method of manufacturing the nozzle includes using photolithographic processing and etching. Another method of manufacturing the nozzle includes, for example, laser drilling. These methods are well known in the art and will be appreciated by one of ordinary skill in the art, therefore, further explanation is not necessary. Moreover, it will be appreciated by one of ordinary skill in the art that nozzle 114 can be tapered, conical, straight, of complex shape, or the like.

According to another embodiment, wherein the device is configured with an array of microjets 204 and an array of nozzles 214, as shown in FIG. 2A for example, multiple injectate substances can be delivered through different nozzles. According to such an embodiment, each microjet 204 can be in communication with different reservoirs or different groupings of microjets 204 can be in communication with different reservoirs such that some microjets can inject a particular injectate while other microjets injects another injectate.

According to yet another embodiment with an array of microjets 204, FIG. 2B for example, each microjet can be a discrete delivery unit 242. Accordingly, each delivery unit 242 can be individually actuated by microprocessor 206. Furthermore, microprocessor 206 can be programmed to operate a single delivery unit 242 at a time until the particular injectate contained within that delivery unit is exhausted, then initiate operation of a next delivery unit until the injectate from each delivery unit is exhausted.

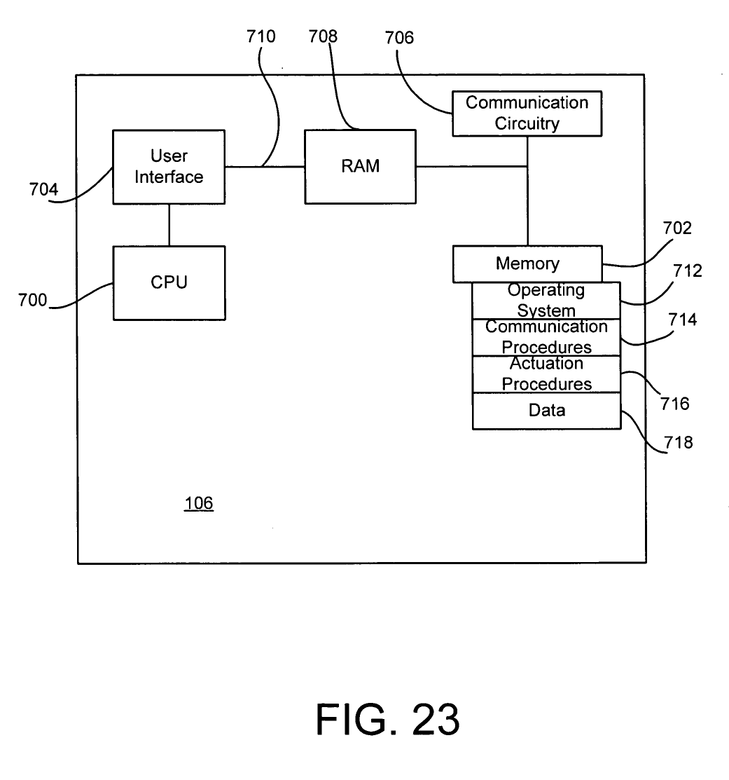

A preferred microprocessor 106 will now be described. As shown in FIG. 23, microprocessor 106 comprises a central processing unit (CPU) 700, a memory 702, user interface 704, communications interface circuit 706, a random access memory (RAM) 708, and a bus 710 that interconnects these elements. Microprocessor 106 is programmable and stores data relating to the administration regime of a particular injectate, a patients requirements, microjet actuation patterns, reservoir mixing times and/or conditions, dosage requirements, and the like, in memory 702. The CPU 700 interprets and executes the data stored in memory 702 for administration of injectate 108. Memory 702 also includes actuation procedures 716 for controlling actuation timing and sequence of microjets 104 and thus controlling administration of injectate. In use, depending upon which force generation mechanism 118, as described above, is incorporated in a particular embodiment of the repetitive microjet device 100, microprocessor 106 either controls the delivery of a voltage to the piezoelectric mechanism, a voltage to electrodes to cause vaporization of actuation fluid, controls an electromagnet, movement of high pressure gas, or the like to control actuation of microjet 104. Throughout this specification the microprocessor 106 is referred to as controlling activation of the microjets. It will be appreciated by one of ordinary skill in the art that the microprocessor controls the supply of power to the force generation mechanism of the microjet. For example, the microprocessor can activate a switch, such as a transistor, which causes power to flow to the force generation mechanism from the power supply, thereby activating the force generation mechanism. However, for convenience to the reader, this process will be referred to as the microprocessor controlling activation of the microjet.

Microprocessor 106 can be programmed to control the activation of the microjet to deliver a certain dosage of treatment to a patient at specified intervals over a specified time period. At the appropriate time, microprocessor 106 will initiate actuation of microjets 104 to ‘fire’ or actuate and deliver the prescribed treatment(s). Therefore, a patient can benefit from a system that maintains optimal dosage levels in the systemic system throughout the day and night automatically (without further human intervention), such that the treatment may have an optimal effect on the patients condition. Moreover, because delivery or injection with the jet of injectate only penetrates the stratum corneum and delivers the treatment into the epidermis, where there is no nerve endings, the process is painless to the user. Microprocessor 106 can also control the destruction of reservoir dividers 320, FIG. 5, between independent chambers within reservoir 102, as described above, for timely mixing of injectate components.

According to another embodiment, memory 702 of microprocessor 106 maintains a record of the quantity of injectate delivered, timing of administration, number of administrations, and the like for future analysis and evaluation to improve the treatment regime for patients.

In an alternative embodiment, microprocessor 106, can also include a user interface 704. User interface device 704 can be a button, switch, or other mechanism which can be activated by the user to stimulate an administration of injectate at any given time. For example, a boost button 136 can be positioned such that it is in communication with microprocessor 106 through a booster button communication link 138. Accordingly, if a patient or administrator determines a need to deliver a treatment dosage of injectate at any given time the boost button 136 can be activated, thereby bypassing the programmed administration regime and delivering an on-demand predetermined dosage of injectate. This can be advantageous for an embodiment where the device is used to deliver pain medication because the need for pain medication can arise outside of a predetermined delivery regime. However, associated with user interface device 704, microprocessor 106 can be pre-programmed with a safety feature such that the user can only trigger the user interface device 704 as many times in a given period, such that, a patient will not overdose or abuse an injectate. The number of times a patient can activate the user interface device 704 can be adjustable depending on what substance comprises the injectate, the age of the patient, the weight of the patient, the severity of the condition of the patient, or the like.

According to yet a further embodiment, microprocessor 106 has communications interface circuitry 706 to communicate with another computer system. A doctor, researcher, or the like can interface with microprocessor 106 through a computer, handheld computer, wireless connection, or the like and access information regarding the frequency of administration, dosage delivered at each interval, variety in dosage delivered, total dosage delivered, and the like. Furthermore, the doctor or researcher can download data 718 saved in memory 702 or modify the administration regime or activation procedures 716. Interfacing with microprocessor 106 can be useful to the continued understanding of treating certain conditions and the development of new and better treatment substances and regimes.

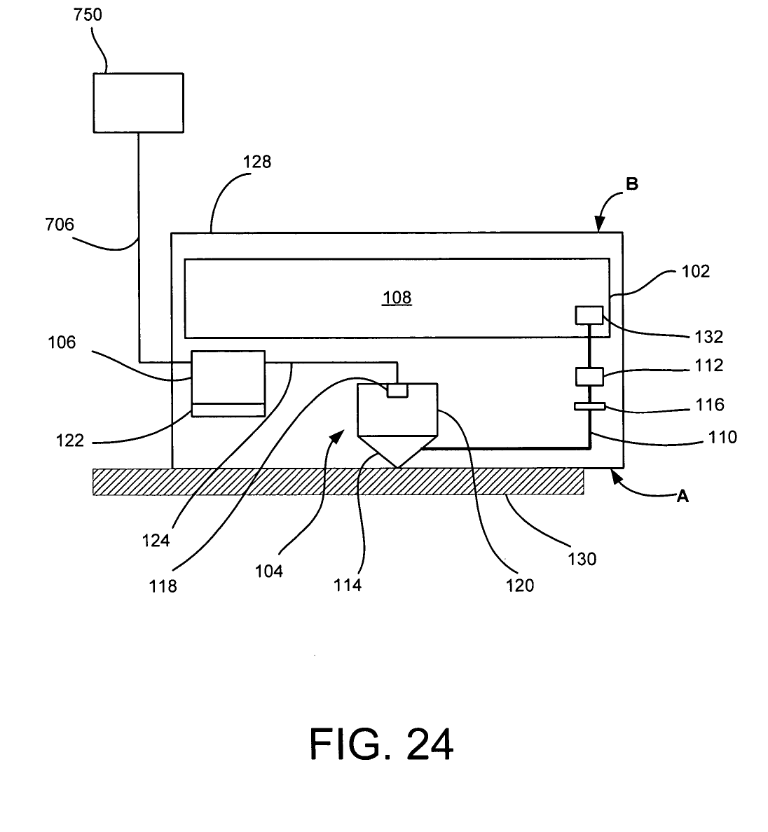

In an alternative embodiment, as shown in FIG. 24, microprocessor 106 is in communication with biosensor 750. Biosensor 750 can be implanted into the body of the user or can be external to the user. Biosensor 750 is preferably a sensor for sensing a biological condition that injectate is designed to treat, circumvent, alter, cure, augment, or the like. Biosensor 750 is in communication with microprocessor 106 through communication device 706, which can be a hard-wire connection, wireless, or the like. Biosensor 750 preferably takes measurements of biological conditions and sends the measurements to microprocessor 106 through communication device 706. Microprocessor 106 reads the measurements taken by biosensor 750, and in response to conditions within a certain predetermined parameter range, microprocessor 106 will actuate microjet 104 to inject injectate to the user for treating the sensed condition.

According to another embodiment, the device 100 can include a condition sensor 133, FIG. 1. Condition sensor 133 is preferably configured to sense whether the device 100 is in contact or otherwise in position with respect to biological barrier 130 such that activation of the device 100 will result in injection of the injectate. If the device 100 is removed from the biological barrier 130 or otherwise out of position, the activation of microjet 104 may be ineffective and not result in administration of injectate into the patient. Therefore, condition sensor 133, being in communication with microprocessor 106, can provide feedback indicating whether microjet 104 should be activated or restrained from activation until the device 100 is re-positioned. Furthermore, condition sensor 133 can include a buzzer or other type of alarming mechanism to notify the patient or attending person(s) that the device is out of position and restrained from activation. The condition sensor can be, for example, a temperature sensor, a pressure sensor, or the like. In an alternative embodiment, the sensor 133 can be configured to sense a pressure generated by the force generation mechanism, thereby, providing a feed-back mechanism to the microprocessor for monitoring functionality of the force generation mechanism.

Microprocessor 106, FIG. 1, controls the injection energy, injection speed, injection volume per ejection of injectate ejected from microjet, drug volume delivery profile over time, and the like. Furthermore, microprocessor 106 can be programmed to deliver a dosage volume that is variable with time to maximize therapeutic benefit. This can be particularly critical for certain conditions which require circadian variation or pulsatile delivery, such as with human growth hormone (hGH) for Growth Hormone Deficiency (GHD), insulin delivery for meal-time blood glucose level management for diabetics, and the like.

Referring back to FIG. 1, repetitive microjet device 100 also includes a power supply 122. Power supply 122 can be a battery such as an NiCd, NiMH, LiMnO₂ battery, disposable battery, rechargeable battery, or the like. Preferably a lightweight, dimensionally small, long lasting, inexpensive, and disposable battery comprises power supply 122. However, in an alternative embodiment, power supply 122 could be another acceptable form of power supply to provide a voltage for the force generation mechanism 118 and microprocessor 106.

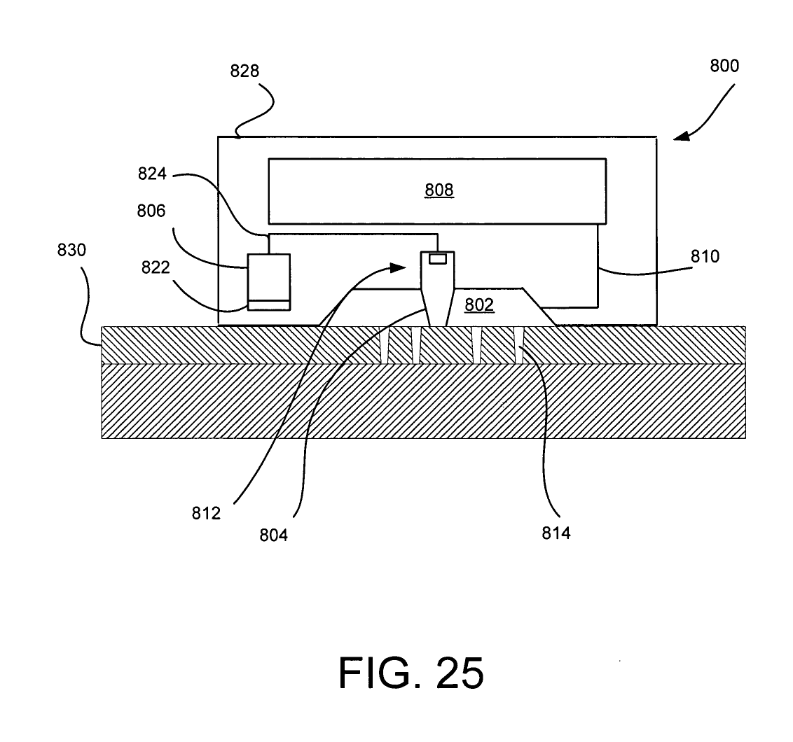

According to an alternative embodiment, as shown in FIG. 25, the transdermal microjet device 800 includes an external reservoir 802. External reservoir 802 is configured as a recessed reservoir adjacent nozzle 804. Accordingly, external reservoir 802 can be filled with a substance to be transferred across a biological barrier 830. The substance to be transferred across biological barrier 830 can be delivered to external chamber from reservoir 808 through a feed line 810. In use, the transdermal microjet device 800 is positioned adjacent biological barrier 830 and the microjet 812 is actuated, as described above. Upon actuation, microjet 812 ejects a jet of solution, thereby piercing biological barrier 830 and creating pores 814. As transdermal microjet device 800 is move with respect to biological barrier 830, the pores 814 generated by the jet of substance from microjet 812 are left available for the substance in external reservoir 802 to passively diffuse through. Furthermore, a substrate can be added to the substance to be transferred across biological barrier 830 to assist in increasing the permeability of biological barrier 830.

Alternatively, transdermal microjet device 800, as shown in FIG. 25, can be utilized for sampling, collecting bodily fluids, taking diagnostic readings of biological specimens through a biological barrier 830, or the like. In such a configuration, microjets 812 are actuated, as described above, and typically inject a saline type solution into biological barrier 830, however, it will be appreciated by one of ordinary skill in the art that any suitable solution for ejection through microjet 812 and into biological barrier 830 can be utilized. Following injection into biological barrier 830, biological fluids diffuse out of pores 814 generated by the injection jet. This biological fluid can then be collected and sampled or analyzed. In an alternative embodiment, the microjets 812 can contain an analyte for injection into the biological tissue. Following injection of the analyte, the analyte can be detected or measured through typical optical or fluorescence techniques. It will be appreciated by one of ordinary skill in the art that many other chemical, biochemical, and/or biological diagnostic techniques

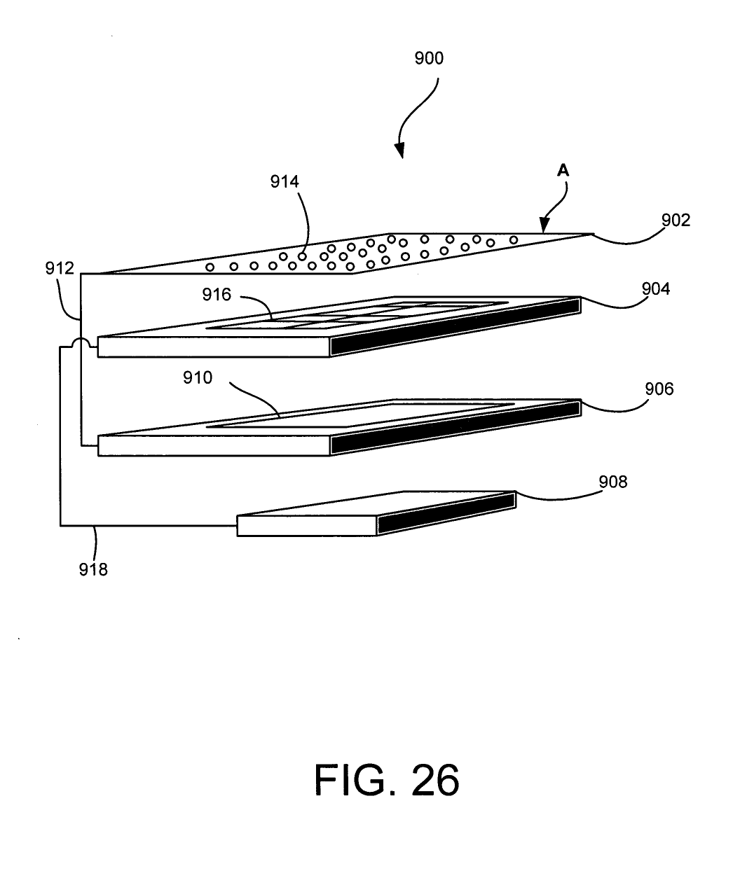

According to a preferred embodiment of the present invention the transdermal microjet device is configured as a drug delivery patch 900, as shown, for example, in FIG. 26. Drug delivery patch 900 is preferably constructed from laminate layers 902, 904, 906, and 908 of biocompatible and drug inert material such as polydimethylsiloxane (PDMS), polyethylene, polyethyleneterephthalate (PET), fluoropolymers, or the like.

The microjet layer 902, control circuitry layer 904, and reservoir layer 906 typically comprise the administration unit which is preferably disposable following complete administration of the drug components. While a microprocessor 908 is housed in a microprocessor layer which is not necessarily disposable and adapted to interact with the administration unit such that a patient can retain the microprocessor layer 908 and re-connect it to a new administration patch. As shown in FIGS. 11 and 15, the administration unit 102 and 462, respectively, can be detached from the microprocessor unit 106. According to this embodiment, a control unit includes the microprocessor 106 and the force generation mechanism 410 and 450, respectively. Therefore, by retaining the control unit when replacing the administration unit, the microprocessor and the force generation mechanism are both retained, such that the disposable portion of the device is limited to the administration unit. As a result, the replacement cost of the administration unit can remain low and the manufacturing processes efficient.

Reservoir layer 906 preferably includes a recessed area 910 which, when coupled with control circuitry layer 904 forms a reservoir for storing injectate components. Reservoir layer 906 is fluidly coupled with microjet layer 902 through feed line 912 for maintaining microjets 914 supplied with injectate. Control circuitry layer 904 includes the electrical circuitry 916 that activates microjets 914. Surface A, the proximal surface of microjet layer 902 preferably includes an adhesive for adhering transdermal drug delivery patch 900 to the skin of a user.

Microprocessor layer 908 typically includes microprocessor 106 and can include power supply 122. Microprocessor layer 908 is configured to house the microprocessor 106 for controlling the actuation of microjets 914. Microprocessor layer 908 is electrically coupled with control circuitry layer 904 through control line 918. Preferably, microprocessor layer 908 is configured to be removably attachable to the administration patch such that microprocessor 106 can be retained after the injectate 108 of administration patch is completely expelled or administration of a particular injectate is complete. Accordingly, a patient can then receive a renewed administration patch with further injectate to be administered and microprocessor 908 can be affixed thereto such that administration of the injectate can continue as earlier programmed for the particular patient or treatment regime.

The power supply 122 may be housed in the administration patch or in the microprocessor layer 908. When the power supply is housed in the administration patch, it is configured to be disposed of with the administration patch following completion of treatment. Therefore, in this configuration, each time the user receives a new administration patch, a new power supply will be provided, assuring that the power supply will not fail partially through a treatment regime.

In an alternative embodiment, as shown in FIGS. 11 and 15, force generation mechanism 410 and 450, respectively, can be configured as a component of microprocessor layer 908 such that the mechanism is retained when administration patch is disposed of, thus, increasing efficiency and reducing cost to the end user.

Preferably the laminate layers are bound together. The laminate layers can be bound together with a chemical bond, thermal bond, or the like. Furthermore, it is desirable that the patch be constructed in an efficient economical form and be disposable following administration of the contents.

Laminate layers 902, 904, and 906 are preferably constructed from a flexible, biocompatible, drug inert material such that drug delivery patch 900 can be applied to a position on a human body and conform to the contour of the body. Furthermore, because transdermal drug delivery patch 900 is flexible it does not restrict activity of the user. According to an alternative embodiment, transdermal drug delivery patch 900 can be constructed from material that is not flexible. Therefore, the transdernal drug delivery patch 900 does not contour to the position of application.

The transdermal microjet device 100 can be configured as a transdermal drug delivery system that is applied to the skin of the user by an adhesive. In alternative embodiments, the device can be positioned in contact with the skin, affixed in place by belt, a buckle, adjustable bands such as elastic bands, or the like.

According to an alternative embodiment, transdermal microjet device 100 can be configured as a hand held or robot held applicator of drugs, treatment solutions, saline solutions, or the like for treating a biological disorder, injury, disease, condition, or the like. Alternatively, the transdermal microjet device can be configured as an implantable device that interfaces with internal organs, tumors, biological barriers such as the dura mater and pia mater, or the like. Furthermore, the transdermal microjet device can be configured as a long term implantable sustained controlled drug release mechanism. The implantable mechanism can be controlled wirelessly from external to the implant site for altering the programmed treatment regime. The device, as described above, can also be utilized in place of an intravenous drug delivery system. In this embodiment, the device can be used to deliver the drug transdermally into the epidermis. The device can be placed on the patient's skin and the device reservoir can be a traditional intravenous (IV) drug drip supply, for example. The drug diffuses from the epidermis into the vein in a very short period of time that can be tolerated in a large number of IV drug delivery application. Furthermore, in patients needing sustained intravenous treatments, complications often arise in relation to the implant site of the catheter. Also, the site of catheter insertion is a prime route for infection to enter the body. The use of the present invention according to this embodiment reduces the chance for infection and other complications from the traditional intravenous drug delivery systems.

Because the present invention is directed to a mechanism and methods for mechanical delivery of drugs to a biological tissue the mechanism is applicable to drugs irrespective of their physicochemical properties such as partition coefficient, solubility, charge, molecular weight, and the like. However, it will be appreciated by one of ordinary skill in the art that a substance can be added to the injectate to increase permeability of the skin. Such a substance can be a chemical surfactant or the like.

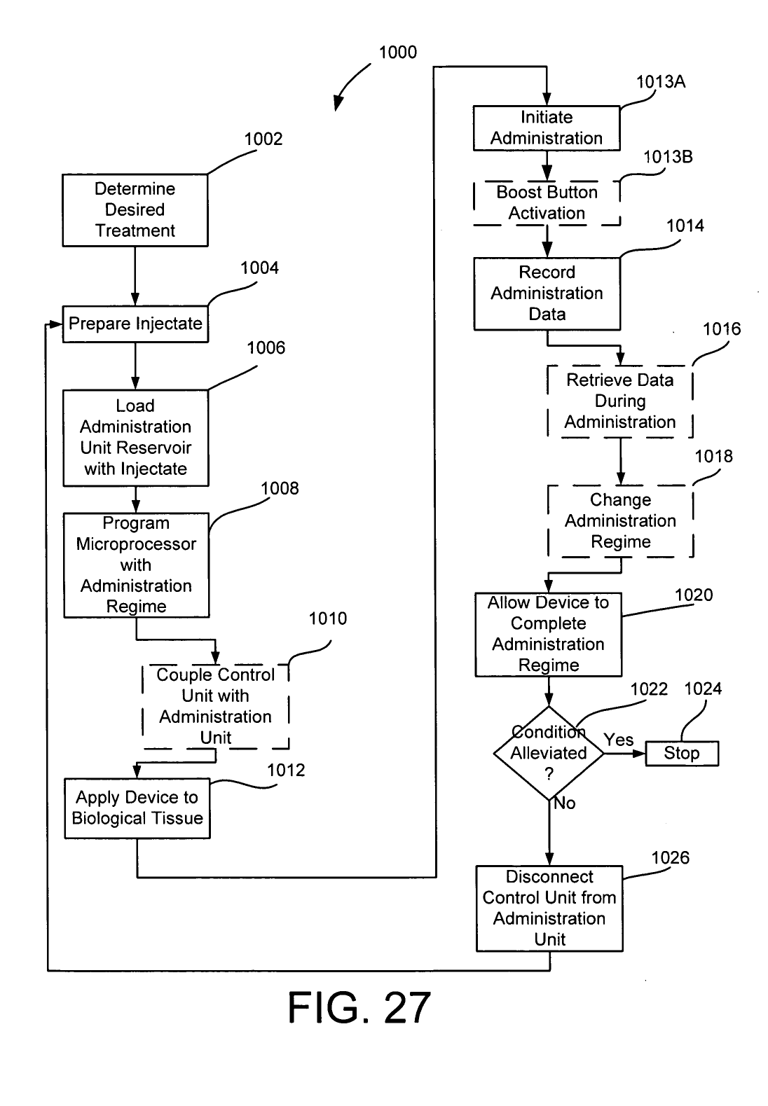

FIG. 27 shows methods for using the present invention as a drug delivery device. According to the method shown, the method begins with a diagnosis of a condition and an associated choice of a desired treatment for that condition, at step 1002. Once a treatment has been selected, an injectate 108 is prepared. The injectate is the substance that will be administered using the transdermal microjet device 100 of the present invention. The injectate can be a drug, antibiotic, pain reliever, placebo, saline, or the like. Next, at step 1006, the injectate is loaded into the reservoir 102 of the transdermal microjet device 100. The microprocessor 106 is then programmed with a preferred administration regime for the particular condition and treatment chosen, at step 1008. Next, if the microprocessor 106 is separate from the administration unit of the transdermal microjet device 100, then the two components are coupled together, at step 1010. The coupling of the microprocessor 106 and the administration unit can be a pin wire connection, wireless connection, or the like. The device is applied to the biological tissue to be treated at step 1012. The biological tissue the device is coupled to may be the tissue to be treated, such as applying the device directly to a tumor to treat the tumor, or the biological tissue may be a barrier the injectate must cross to reach the tissue to be treated. For example of the later, if the desired utility of the device is to deliver a drug systemically to a patient, the skin may be the biological barrier to be crossed. Therefore, the device is coupled to, or brought in contact with the biological tissue and the device injects the injectate across the barrier for systemic delivery of the injectate.

During administration of the injectate to the biological tissue, data relating to the administration of the injectate is recorded, at step 1014. Data that may typically be recorded includes the time of each administration, the quantity of each administration, and the like. In an alternative embodiment, the device may include sensors, such as biosensors that monitor and record biological activity of the patient, such as temperature, blood pressure, pulse, blood glucose levels, or other such biological and/or chemical conditions of the patient. Next, if the physician or researcher in charge of the biological tissue being treated wishes, they can electronically interface with the device and receive the data that is being recorded in real time and/or at any time during administration of the injectate, as shown at step 1016. The physician or researcher can also change the administration regime through the electronic interface with microprocessor 106 during the administration period of the injectate, at step 1018. Next, the administration of the injectate is allowed to carry out the administration regime, at step 1020.

If, following the complete administration of an injectate, the condition is alleviated, then the method terminates at step 1024. However, if following complete administration of an injectate, the condition is not alleviated, then the microprocessor 106 is disconnected from the administration unit, the administration unit is discarded, and the microprocessor is retained, at step 1026. A new injectate is prepared, at step 1004, and the treatment method continues as previously described. The new injectate can be another quantity of the same injectate previously administered or a different injectate composition can be administered.

An exemplary description of the performance of the steps of FIG. 27 is given below. For example, an administrator or physician would typically perform step 1002, determining a desired treatment for the patient. Steps 1004, 1006, and 1008 can typically be performed during manufacture of the device such that each administration unit is prepared and sealed prior to shipping to a dispenser, such as a pharmacy. Step 1010, coupling the control unit with the administration unit can be performed by either the patient, pharmacist, physician, or the like, as is similar with step 1012. The device can be applied to the patient by either the patient, pharmacist, physician, or the like. Typically, initiation of administration begins following application of the device to the patient, at step 1013A and the boost button can be activated by either a patient, pharmacist, physician, or the like, at step 1013B, to administer an on-demand delivery of injectate. The steps of retrieving data, step 1016, and changing an administration regime, step 1018, are typically performed by the physician or a technician upon the direction of a physician. Upon completion of administration of an administration unit, the patient most often will disconnect the control unit from the administration unit, step 1026, and replace the used administration unit with a new administration unit, step 1010, however, a physician or other medical specialist can perform this step. It will be appreciated by one of ordinary skill in the art that this recitation of operation of the steps of the present invention are for explanatory purposes and not meant by way of limitation. The present invention can be performed by a patient, an administrator, during manufacture, or a combination of the above, whichever suits a particular situation, proves efficient and convenient, and facilitates treatment of a medical condition.

What is claimed is:

1. An active, fluid delivery system, comprising:
a support structure;
at least one exit orifice defined in the support structure, said orifice having a diameter between about 1 μm and about 500 μm;
a fluid reservoir configured to contain a fluid to be delivered to a tissue and communicating with said at least one exit orifice; and
repeatable activation means cooperating with said fluid reservoir and said at least one exit orifice for ejection of fluid in response to an activation signal.

2. The delivery system of claim 1, wherein said fluid reservoir and said repeatable activation means are disposed in the support structure.

3. The delivery system of claim 1, wherein said support structure is adapted to be in contact with a skin surface with the at least one exit orifice adjacent the skin surface.

4. The delivery system of claim 1, wherein the support structure includes a nozzle defining said orifice, said nozzle being configured and dimensioned to accelerate the fluid exiting therefrom.

5. The delivery system of claim 1, further comprising a controller in communication with the repeatable activation means, the controller being capable of generating the activation signal.

6. The delivery system of claim 5, wherein said controller is a microprocessor programmable to control a patterned administration regime.

7. The delivery system of claim 6, wherein the sustained administration regime occurs over a time period of not less than about 500 ms and not more than about 10 days.

8. The delivery system of claim 4, wherein, prior to ejection, the nozzle is configured to maintain the fluid remote from the tissue a substantially fixed distance.

9. The delivery system of claim 8, wherein the fixed distance spaces the fluid, prior to ejection of the fluid, not more than about 5000 μm from the tissue.

10. The delivery system of claim 1, further comprising an array of exit orifices defined in the support structure and in communication with the fluid reservoir.

11. The delivery system of claim 1, wherein the fluid reservoir includes a storage reservoir configured to store fluid.

12. The delivery system of claim 11, further comprising a pressurization mechanism for pressurizing the stored fluid in the storage reservoir.

13. The delivery system of claim 11, wherein the storage reservoir is divided into multiple storage reservoirs by a reservoir divider.

14. The delivery system of claim 13, further comprising an array of exit orifices defined in the support structure, wherein a first exit orifice is in communication with a first storage reservoir storing a first fluid such that the first fluid can be ejected through the first exit orifice and at least a second exit orifice is in communication with at least a second storage reservoir storing a second fluid such that the second fluid can be ejected through the second exit orifice.

15. The delivery system of claim 13, further comprising a reservoir divider disruption mechanism configured and dimensioned to disrupt the reservoir divider prior to administration of a substance contained in the reservoir.

16. The delivery system of claim 15, wherein said reservoir divider disruption mechanism is a piezoelectric mechanism.

17. The delivery system of claim 1, further comprising:
a sensor for sensing if a condition is satisfied; and
a control unit configured to produce the activation signal to actuate the repeatable activation means upon receiving a signal from the sensor that the condition is satisfied.

18. The delivery system of claim 1, further comprising:
a control unit configured to produce the activation signal to actuate the repeatable activation means; and
a sensor for sensing if a condition is satisfied and, if so, communicating a signal to the control unit to not produce the activation signal, thereby, not actuating the repeatable activation means.

19. The delivery system of claim 17, wherein the sensor is located remotely from the support structure.

20. The delivery system of claim 17, wherein the sensor is implanted into a patient.

21. The delivery system of claim 17, wherein the sensor is capable of sensing a biological condition of a patient.

22. The delivery system of claim 17, wherein said sensor is coupled with said support structure such that said sensor determines conditions related to administration.

23. The delivery system of claim 22, wherein said sensor is a temperature sensor for determining if said support structure is positioned adjacent the tissue.

24. The delivery system of claim 22, wherein said sensor is a pressure sensor for providing a feed-back mechanism for monitoring functionality of the repeatable activation means.

25. The delivery system of claim 1, further comprising an antagonist reservoir configured and dimensioned in communication with the fluid reservoir, integrity of both reservoirs being in cooperation such that upon compromise of the fluid reservoir's integrity, the integrity of the antagonist reservoir is compromised, thereby releasing an antagonist component from the antagonist reservoir capable of inactivating the fluid.

26. The delivery system of claim 1, further comprising a power supply for supplying a drive force for the activation signal and a drive force for the repeatable activation means.

27. The delivery system of claim 1, wherein the repeatable activation means is a piezoelectric mechanism that generates a pressure change in the fluid.

28. The delivery system of claim 1, wherein the repeatable activation means is a phase change mechanism that generates a pressure change in the fluid.

29. The delivery system of claim 1, wherein said repeatable activation means is an electromagnetic mechanism that generates a pressure change in the fluid.

30. The delivery system of claim 1, wherein said repeatable activation means is a high pressure hydraulic mechanism that generates a pressure change in the fluid.

31. The delivery system of claim 1, wherein said repeatable activation means includes multiple explosive mechanisms, each explosive mechanism capable of generating a pressure change in the fluid upon detonation of said explosive mechanism.

32. The delivery system of claim 1, further comprising a user interface in communication with said repeatable activation means, said user interface being configured to initiate the activation signal in response to manipulation of said user interface.

33. The delivery system of claim 1, wherein the fluid is to be delivered transdermally across epithelial tissue.

34. The delivery system of claim 1, wherein said repeatable activation means generates a pulse width of not less than about 5 ns and not more than about 10 μs.

35. The delivery system of claim 1, wherein a frequency of the repeatable activation means and a duty cycle and length of ejection of fluid are controlled by a control unit.

36. The delivery system of claim 1, further comprising a memory for storing a delivery profile and delivery history of the fluid delivered to the tissue.

37. The delivery system of claim 1, wherein the fluid includes an analyte for delivery to the tissue and subsequent diagnoses of a biological condition.

38. An active, fluid delivery system, comprising:
a support structure;
a fluid ejection chamber within the support structure;
at least one exit orifice defined in the support structure and communicating with the fluid ejection chamber; and
activation means disposed in the fluid ejection chamber,
wherein said fluid ejection chamber, at least one exit orifice, and activation means are configured and dimensioned together for continuously cyclic repeatable ejection of fluid in the range of about 1 pl to about 800 nl.

39. The delivery system of claim 38, wherein said support structure is adapted to be in contact with a skin surface with the at least one exit orifice adjacent the skin surface.

40. The delivery system of claim 38, wherein the support structure includes a nozzle in communication with the fluid ejection chamber and the nozzle has an exit orifice configured and dimensioned to accelerate the fluid exiting therefrom.

41. The delivery system of claim 38, further comprising a controller in communication with the activation means for delivering an activation signal to the activation means.

42. The delivery system of claim 40, wherein, prior to ejection, the nozzle is configured to maintain the fluid remote from a biological tissue a substantially fixed distance.

43. The delivery system of claim 38, further comprising:
a control unit configured to produce an activation signal to actuate the activation means; and
a sensor for sensing if a condition is satisfied and, if so, communicating a signal to the control unit to produce the activation signal and actuate the activation means.

44. An active, fluid delivery system, comprising:
a support structure;
a fluid ejection chamber within the support structure configured to contain a fluid to be delivered;
at least one exit orifice defined in the support structure and communicating with the fluid ejection chamber;
activation means disposed in the fluid ejection chamber; and
a controller programmed to activate the activation means to deliver a sustained repetitive administration regime from not less than about 500 ms to not more than about 10 days.

45. The delivery system of claim 44, wherein said support structure is adapted to be positioned adjacent a biological tissue with the at least one exit orifice adjacent the biological tissue.

46. The delivery system of claim 44, wherein the support structure includes a nozzle in communication with the fluid ejection chamber, the nozzle having an exit orifice configured and dimensioned to accelerate the fluid exiting therefrom, and wherein the nozzle is configured to maintain the fluid remote from a biological tissue a substantially fixed distance prior to ejection.

47. The delivery system of claim 44, further comprising an antagonist reservoir configured and dimensioned in association with the fluid ejection chamber such that prior to the fluid ejection chamber rupturing, the antagonist reservoir ruptures and releases an antagonist component into the fluid to inactivate the fluid.

48. An active, fluid delivery system, comprising:
a support structure;
a fluid reservoir within the support structure configured to contain a fluid to be delivered;
at least one exit orifice defined in the support structure and communicating with the fluid reservoir; and
a piezoelectric mechanism that mechanically deform upon application of a voltage, the piezoelectric mechanism being disposed within the support structure in cooperation with the at least one exit orifice for ejection of fluid in response to an activation signal.

49. An active, transdermal delivery system, comprising:
a support structure;
a fluid reservoir within the support structure configured to contain a fluid to be delivered;
at least one exit orifice defined in the support structure and communicating with the fluid reservoir; and
a phase change mechanism that vaporizes an actuation fluid in response to an activation signal, thereby generating a pressure change in the fluid and ejecting fluid from the exit orifice.

50. An active, transdermal delivery system, comprising:
a support structure;
a fluid reservoir within the support structure configured to contain a fluid to be delivered;
at least one exit orifice defined in the support structure and in communication with the fluid reservoir; and
a phase change mechanism that vaporizes at least some of the fluid in response to an activation signal, thereby generating a pressure change in the fluid and ejecting a portion of the fluid from the exit orifice.

51. The delivery system of claim 50, further comprising a flexible membrane dividing the fluid reservoir into a first compartment and a second compartment, wherein said first compartment contains an actuation fluid and said second compartment contains the fluid to be delivered.

52. The delivery system of claim 50, further comprising an actuation fluid positioned near said phase change mechanism, the actuation fluid being immiscible with the fluid to be delivered.

53. An active, transdermal delivery system, comprising:
a support structure;
a fluid reservoir within the support structure configured to contain a fluid to be delivered;
at least one exit orifice defined in the support structure and communicating with the fluid reservoir; and
an electromagnetic mechanism disposed within the fluid reservoir in cooperation with the at least one exit orifice for ejection fluid in response to an activation signal.

54. An active, transdermal delivery system, comprising:
a support structure;
a fluid reservoir within the support structure configured to contain a fluid to be delivered;
at least one exit orifice defined in the support structure and communicating with the fluid reservoir; and
a high pressure hydraulic mechanism disposed within the support structure in cooperation with the at least one exit orifice for ejection of fluid in response to an activation signal.

55. An active, fluid delivery system having a support structure configured and dimensioned with an array of administration chambers, wherein each administration chamber comprises:
a exit orifice defined in the support structure, said exit orifice having a diameter between about 1 μm and about 500 μm;
a fluid reservoir configured to contain a fluid to be delivered to a tissue, wherein said fluid reservoir is in communication with said exit orifice; and
a repeatable activation means cooperating with said fluid reservoir and said exit orifice for ejection of fluid in response to an activation signal.

56. A method for active, transdermal delivery, comprising:
positioning a support structure defining an orifice adjacent a biological tissue, said orifice having a diameter of between about 1 μm and about 500 μm, and said support structure in fluid communication with a fluid to be administered to the biological tissue; and
actively and repetitively ejecting fluid into the biological tissue.

57. The method of claim 56, further comprising, before said positioning, programming a controller with a sustained administration regime according to a substance to be delivered and a patients needs.

58. The method of claim 56, further comprising, during active and repetitive ejection of said fluid, recording administration data and associated patient data throughout an administration regime.

59. The method of claim 58, further comprising interfacing with the controller to retrieve the administration data during the administration regime.

60. The method of claim 57, further comprising interfacing with the controller to change the administration regime.

61. The method of claim 56, wherein said fluid includes an analyte for performing a diagnosis on a biological system of said biological tissue.

62. The method of claim 56, further comprising, after actively and repetitively ejecting the fluid, extracting fluid from said biological tissue for analysis.

63. The method of claim 56, wherein said actively and repetitively ejecting fluid into the biological tissue is configure to disrupt a stratum corneum of an epithelial tissue such that biological fluid below the stratum corneum can be removed from said biological tissue.
